(19) United States
(12) Patent Application Publication
(10) Pub. No.: US 2008/0305500 A1
(43) Pub. Date: Dec. 11, 2008

(54) NOVEL CELL-BASED ASSAYS FOR IDENTIFYING ENHANCERS OR INHIBITORS OF T1R TASTE RECEPTORS (T1R2/T1R3 SWEET) AND UMAMI (T1R1/T1R3 UMAMI) TASTE RECEPTORS

(75) Inventors: Guy Servant, San Diego, CA (US); Poonit Kamdar, Boston, MA (US); Adam Rivadeneyra, San Diego, CA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Senomyx, Inc., San Diego, CA (US)

(21) Appl. No.: 11/782,336

(22) Filed: Jul. 24, 2007

Related U.S. Application Data
(63) Continuation of application No. 11/808,355, filed on Jun. 8, 2007, now abandoned.

(51) Int. Cl. G01N 33/53; G01N 33/567; G01N 33/569

(52) U.S. Cl. 435/7.21; 435/7.2; 435/7.31

(57) ABSTRACT

This invention relates to improved assays for identifying modulators (enhancers or inhibitors) of sweet (T1R2/T1R3) and umami (T1R1/T1R3) taste receptors. These receptors may comprise the endogenous T1Rs e.g., from humans or rodents, or may comprise functional variants such as chimeric taste receptors comprising the extracellular portion of one T1R or a variant or fragment thereof, either T1R1 or T1R2, and the transmembrane portion of another T1R or a variant or fragment thereof, either T1R1 or T1R2, preferably associated with a T1R3 polypeptide and a suitable G protein. The subject assays preferably use endogenous taste or gastrointestinal cells which express T1R taste receptors or recombinant cell which express such T1Rs, for example mammalian cells or Xenopus oocytes. The modulators (enhancers or inhibitors) identified according to the subject assays are useful as taste modulators (which is confirmed in taste tests) and/or as therapeutics for treating conditions such as diabetes, obesity, weight control, fat metabolism, glucose metabolism, insulin metabolism, satiety and/or the release of satiety peptides such as GLP-1.

### RELATED APPLICATIONS

This application relates to U.S. Ser. No. 179,373 filed on Jun. 26, 2002, U.S. Ser. No. 09/799,629 filed Apr. 5, 2001 and U.S. Ser. No. 10/035,045 filed on Jan. 3, 2002. These applications and the T1R sequences disclosed therein are incorporated by reference in their entirety herein. Also, this patent application relates to and claims priority to U.S. Ser. No. 10/569,870 filed on Aug. 6, 2004, and published as US200070104709. This application is incorporated by reference in its entirety herein.

### FIELD OF THE INVENTION

This invention relates to novel and improved assays for identifying compounds that function as sweet or umami taste modulators and/or potential therapeutics, i.e. compounds which potentiate or inhibit the sweet or umami taste elicited by other compounds, e.g., natural and synthetic sweet and umami tastants but which compounds themselves do not elicit a sweet or umami taste. These compounds because T1Rs are expressed in non-taste cells may also be used to treat or prevent conditions such a obesity, diabetes, satiety, weight gain, cachexia, fat metabolism, glucose metabolism and glucose transport, glucose transporter release (e.g., GLUT2 or SGLT1), glucose absorption, and the release of satiety peptides such as GLP-1, Also, the invention provides novel enhancer and inhibitor compounds and therapeutic and food, beverage and other compositions containing.

### BACKGROUND OF THE INVENTION

The taste system provides sensory information about the chemical composition of the external world. Taste transduction is one of the most sophisticated forms of chemical-triggered sensation in animals. At present, the means by which taste sensations are elicited remains poorly understood. See, e.g., Margolskee (1993) BioEssays 15: 645-50; Avenet, et al. (1989) J. Membrane Biol. 112: 1-8. Taste signaling is found throughout the animal kingdom, from simple metazoans to the most complex of vertebrates. Taste sensation is thought to involve distinct signaling pathways. These pathways are believed to be mediated by receptors, i.e., metabotropic or inotropic receptors. Cells which express taste receptors, when exposed to certain chemical stimuli, elicit taste sensation by depolarizing to generate an action potential, which is believed to trigger the sensation. This event is believed to trigger the release of neurotransmitters at gustatory afferent neuron synapses, thereby initiating signaling along neuronal pathways that mediate taste perception. See, e.g., Roper (1989) Ann. Rev. Neurosci. 12: 329-53.

As such, taste receptors specifically recognize molecules that elicit specific taste sensation. These molecules are also referred to herein as “tastants.” Many taste receptors belong to the 7-transmembrane receptor superfamily, which are also known as G protein-coupled receptors (GPCRs) or “serpentine receptors”. See Hoon, et al. (1999) Cell 96: 451; Adler, et al. (2000) Cell 100: 693. Brauner-Osborne, et al. (2007) “Structure, pharmacology and therapeutic prospects of family C G-protein coupled receptors.” Curr Drug Targets 8(1):169-84.

Other tastes are believed to be mediated by channel proteins. G protein-coupled receptors control many physiological functions, such as endocrine function, exocrine function, heart rate, lipolysis, carbohydrate metabolism, and transmembrane signaling. The biochemical analysis and molecular cloning of a number of such receptors has revealed many basic principles regarding the function of these receptors.

For example, in the Family C of G-protein coupled receptors (GPCRs) from humans is constituted by eight metabotropic glutamate (mGlu(1-8)) receptors, two heterodimeric gamma-aminobutyric acid(B) (GABA(B)) receptors, a calcium-sensing receptor (CaR), three taste (T1R) receptors, a promiscuous L-alpha-amino acid receptor (GPRC6A), and five orphan receptors. The family C GPCRs are characterized by a large amino-terminal domain, which bind the endogenous orthosteric agonists. Additionally, allosteric modulators which bind to the seven transmembrane domains of the receptors have also been reported. Brauner-Osborne, et al. (2007) “Structure, pharmacology and therapeutic prospects of family C G-protein coupled receptors.” Curr Drug Targets 8(1):169-84.

In general, upon a ligand binding to a GPCR, the receptor presumably undergoes a conformational change leading to activation of the G protein. G proteins are comprised of three subunits: a guanyl nucleotide binding alpha-subunit, a beta-subunit, and a gamma-subunit. G proteins cycle between two forms, depending on whether GDP or GTP is bound to the alpha-subunit. When GDP is bound, the G protein exists as a heterotrimer: the Galpha-beta-gamma complex. When GTP is bound, the alpha-subunit dissociates from the heterotrimer, leaving a G betagamma complex. When a Galphabetagamma complex operatively associates with an activated G protein-coupled receptor in a cell membrane, the rate of exchange of GTP for bound GDP is increased and the rate of dissociation of the bound Galpha subunit from the Galphabetagamma complex increases. The free Galpha subunit and Gbetagamma complex are thus capable of transmitting a signal to downstream elements of a variety of signal transduction pathways. These events form the basis for a multiplicity of different cell signaling phenomena, including for example the signaling phenomena that are identified as neurological sensory perceptions such as taste and/or smell. U.S. Pat. No. 5,691,188.

Mammals are believed to have five basic taste modalities: sweet, bitter, sour, salty, and umami (the taste of monosodium glutamate). See, e.g., Kawamura, et al. (1987) Introduction to Umami: A Basic Taste; Kinnamon, et al. (1992) Ann. Rev. Physiol., 54:715-31 (1992); Lindemann (1996) Physiol. Rev. 76: 718-66; Stewart, et al. (1997) Am. J. Physiol. 272: 1-26. Numerous physiological studies in animals have shown that taste receptor cells may selectively respond to different chemical stimuli. See, e.g., Akabas, et al. (1988) Science 242: 1047-50; Gilbertson, et al. (1992) J. Gen. Physiol. 100: 803-24; Bernhardt, et al. J. Physiol. 490: 325-36; Cummings, et al. (1996) J. Neurophysiol. 75: 1256-63.

In mammals, taste receptor cells are assembled into taste buds that are distributed into different papillae in the tongue epithelium. Circumvallate papillae, found at the very back of the tongue, contain hundreds to thousands of taste buds. By contrast, foliate papillae, localized to the posterior lateral edge of the tongue, contain dozens to hundreds of taste buds. Further, fungiform papillae, located at the front of the tongue, contain only a single or a few taste buds.

Each taste bud, depending on the species, contains 50-150 cells, including precursor cells, support cells, and taste receptor cells. See, e.g., Lindemann (1996) Physiol. Rev. 76: 718-66. Receptor cells are innervated at their base by afferent nerve endings that transmit information to the taste centers of the cortex through synapses in the brain stem and thalamus. Elucidating the mechanisms of taste cell signaling and information processing is important to understanding the function, regulation, and perception of the sense of taste.

Complete or partial sequences of numerous human and other eukaryotic chemosensory receptors are currently known. See, e.g., Pilpel, Y. and Lancet, D. (1999) Protein Science, 8: 969-977; Mombaerts (1999) Annu. Rev. Neurosci. 22: 487-50. See also, EP 0 867 508 A2, U.S. Pat. No. 5,874,243; U.S. Pat. No. 6,955,887; WO 92/17585; WO 95/18140; WO 97/17444; WO 99/67282.

Additionally of relevance to the present invention, within the last several years, a number of groups including the present assignee Senomyx Inc., have reported the identification and cloning of genes from two GPCR families that are involved in taste modulation and have obtained experimental results that provide a greater understanding of taste biology. These results indicate that bitter, sweet and amino acid taste, also referred as umami taste, is triggered by activation of two types of specific receptors located at the surface of taste receptor cells (TRCs) on the tongue i.e., T2Rs and T1Rs. Gilbertson et al., (2000) Curr. Opin. Neurobiol., 10(4):519-27; Margolskee, (2002) J. Biol. Chem. 277(1):1-4; Montmayeur et al., (2002) Curr. Opin. Neurobiol., 12(4):366-71. It is currently believed that at least 26 and 33 genes encode functional receptors (T2Rs) for bitter tasting substances in human and rodent respectively. Adler et al., (2000) Cell 100(6):693-702; Matsunami et al., (2000) Nature 404(6678):601-4. By contrast there are only 3 T1Rs, T1R1, T1R2 and T1R3, which are involved in umami and sweet taste. Li et al., (2002) Proc. Natl Acad Sci., USA 99(7):4692-6; Nelson et al., (2002) Nature (6877):199-202; Nelson et al., (2001) Cell 106(3):381-96. Structurally, the T1R and T2R receptors possess the hallmark of G protein-coupled receptors (GPCRs), i.e., 7 transmembrane domains flanked by small extracellular and intracellular amino- and carboxyl-termini respectively.

T2Rs which have been cloned from different mammals including rats, mice and humans. Adler et al., (2000) Cell 100(6):611-8. T2Rs comprise a novel family of human and rodent G protein-coupled receptors that are expressed in subsets of taste receptor cells of the tongue and palate epithelia. These taste receptors are organized in clusters in taste cells and are genetically linked to loci that influence bitter taste. The fact that T2Rs modulate bitter taste has been demonstrated in cell-based assays. For example, mT2R-5, hT2R-4 and mT2R-8 have been shown to be activated by bitter molecules in in vitro gustducin assays, providing experimental proof that T2Rs function as bitter taste receptors. Chandrashekar et al., (2000) Cell 100(6): 703.

More particularly, the members of the “T1R” family of taste-cell-specific GPCRs. In particular embodiments of the invention, the T1R family members include rT1R3, mT1R3, hT1R3, rT1R2, mT1R2, hT1R2, and rT1R1, mT1R1 and hT1R1. It is known that the three T1R gene members T1R1, T1R2 and T1R3 form functional heterodimers that specifically recognize sweeteners and amino acids. Li et al., (2002) Proc. Natl Acad Sci., USA 99(7):4692-6; Nelson et al., (2002) Nature (6877): 199-202; Nelson et al., (2001) Cell 106(3):381-96. Functional studies performed in HEK293 cells expressing the promiscuous G protein G_{□□}15/16, also disclosed therein have shown that the rodent and human T1R2/T1R3 combination recognizes natural and artificial sweeteners while the rodent and human T1R1/T1R3 combination recognizes several L-amino acids and monosodium glutamate (MSG), respectively. Li et al., (2002) Proc. Natl Acad Sci., USA 99(7):4692-6; Nelson et al., (2002) Nature (6877):199-202; Nelson et al., (2001) Cell 106(3):381-96. These results, demonstrate that T1Rs are involved in sweet and umami taste.

Particularly, the co-expression of T1R1 and T1R3 in recombinant host cells results in a hetero-oligomeric taste receptor that responds to umami taste stimuli. Umami taste stimuli include by way of example monosodium glutamate and other molecules that elicit a “savory” taste sensation. By contrast, the co-expression of T1R2 and T1R3 in recombinant host cells results in a hetero-oligomeric sweet taste receptor that responds to both naturally occurring and artificial sweeteners. As with T2Rs, T1R DNAs and the corresponding polypeptides have significant application in cell and other assays, preferably high throughput assays, for identifying molecules that modulate T1R taste receptors; particularly the T1R2/T1R3 receptor (sweet receptor) and the T1R1/T1R3 receptor (umami receptor). T1R modulators can be used as flavor-affecting additives in foods, beverages and medicines.

Also, it has been recently reported that taste receptors may be expressed in non-oral tissues, e.g., in the digestive system and potentially other organs such as the kidney. Particularly it has been reported that sweet, umami and bitter taste receptors are expressed in cells other than in the oral cavity such as gastrointestinal cells. (See, e.g., Sternini et al., Amer. J Physiol. Gastrointestinal and Liver Physiology, 292:G457-G461, 2007; Mace, O. J. et al, J. Physiology. 10.1113/jphysiol.2007.130906. Published online May 10, 2007). Also, it has been reported by various groups (Margolskee et al., Bezencon et al., Rozengurt et al, and Sternini et al. (2007) (Id)) that bitter and umami taste receptors and other taste signaling molecules such as TRPM5 and gustducin are expressed in specialized cells in the gastrointestinal tract. (See e.g., Margolskee et al., Genes Brain Behavior 2007 (epub March 21); Rozengurt et al., Amer. J. Physiol. Gastroent. Liver Physiol. 291(2):G171-7 (2006); Bezencon et al., Chem. Senses 32(1):41-47(2007)). Margolskee et al. (Id) further reports that the loss of T1R3 or gustducin in rodents resulted in changes in insulin metabolism and the release of satiety peptides such as GLP-1 (glucagon-like peptide 1). Moreover, it is likely that salty receptors are expressed in the urinary tract. Based on these observations, taste receptors have been suggested to be involved in functions not directly related to taste such as digestive functions for example food sensing, insulin and/or glucose metabolism and absorption, glucose and/or insulin release and transport, gastric motility, food absorption, the release of satiety peptides such as glucagon-like peptide and the expression of glucose transporters such as GLUT2 and SGLT1.

Although much is known about the psychophysics and physiology of taste cell function, very little is known about the molecules and pathways that mediate its sensory signaling response. The identification and isolation of novel taste receptors and taste signaling molecules could allow for new methods of chemical and genetic modulation of taste transduction pathways. For example, the availability of receptor and channel molecules could permit the screening for high affinity agonists, antagonists, inverse agonists, and modulators of taste activity. Such taste modulating compounds could be useful in the pharmaceutical and food industries to improve the taste of a variety of consumer products, or to block undesirable tastes, e.g., in certain pharmaceuticals.

Therefore, a need exists for novel and improved assays for identifying T1R and T2R agonists and antagonists are still needed, especially for T1R2 and T1R3. In particular other high throughput assays that provide for the rapid and accurate identification of T1R or T2R agonists and antagonists would be beneficial. Also, a greater understanding of what conditions and materials yield functional T1Rs and T2Rs and assays based on this greater understanding would further be beneficial.

### BRIEF SUMMARY OF THE INVENTION

It is an object of the present invention to provide methods for identifying taste modulating compounds, particularly sweet and umami taste modulating compounds. Preferably, such methods may be performed by using a combination of any one or all members of a family of mammalian G protein-coupled receptors, herein referred to as “T1Rs”, active in taste perception as hetero-oligomeric complexes, or fragments or variants thereof, and genes encoding such T1Rs, or fragments or variants thereof, disclosed herein.

These and other objects of the invention are met by one or more of the following embodiments.

The invention provides novel and improved assays for identifying compounds which function as enhancers or inhibitors of the sweet (T1R2/T1R3) or umami (T1R1/T1R3) taste receptor, i.e., compounds which potentiate sweet or umami taste elicited by other compounds and/or which are useful as therapeutics e.g., in treating, preventing, or modulating obesity, weight management, diabetes, fat metabolism and/or absorption, glucose metabolism and/or absorption, insulin release and/or metabolism, satiety, satiety peptide release such as GLP1 release, and/or glucose transporter release such as GLUT2 or SGLT1.

More specifically the invention provides novel and improved assays, preferably fluorimetric calcium imaging assays, for identifying enhancers or inhibitors of the sweet (T1R2/T1R3) taste receptor or the umami (T1R1/T1R3) taste receptor that are more sensitive than prior assays and which thereby facilitate the detection of enhancer or inhibitor compounds which were undetected by prior assay methods.

The invention further provides novel enhancers or inhibitors identified by such methods and compositions containing that modulate (enhance) the sweet or umami taste elicited by other compounds.

More specifically the invention provides novel cell based assays whereby (i) a test cell that expresses the umami or sweet taste receptor and which comprises a means for detecting the activation of the sweet or umami taste receptor (such as a voltage or calcium sensitive indicator fluorescent dye or label) is preincubated with an amount of one or more potential enhancer compounds for a time (preincubation time) prior to (ii) contacting said same test cell with a suboptimal concentration of a known sweetener or umami compound, (iii) assaying the activity of the sweet or umami taste receptor after step (i) and (ii), (iv) further assaying the activation of said sweet or umami taste receptor in the same test cell the presence of the same suboptimal concentration of the known umami or sweet compound but in the absence of said preincubation with said potential enhancer compound, and comparing the activity detected in step (iii) and (iv) and if (iii) is respectively greater or lesser than (iv) identifying said compound as a putative enhancer or inhibitor of said known sweet or umami compound. (This putative enhancer or inhibitor activity can potentially be confirmed e.g., in suitable taste tests or in therapeutic screening methods).

Still more specifically, it is an object of the invention to provide novel enhancer or inhibitor assays which comprise the following steps (i) loading a suitable test that expresses the T1R2/T1R3 sweet receptor or the T1R1/T1R3 taste receptor with a dye or label that permits the detection of changes in a parameter that correlates to sweet or umami receptor activity such as changes in intracellular calcium or cell voltage; (ii) preincubating said loaded test cell with at least one compound which is to be screened for its efficacy as a specific sweet or umami enhancer or inhibitor for a specific time (preincubation time); (iv) contacting said loaded test cell after said preincubation step with a suboptimal amount of at least one sweet or umami compound; (v) further contacting another dye or label loaded test cell with substantially the same suboptimal concentration of the same at least one sweet or umami compound but without said initial preincubating step with said at least one potential sweet or umami enhancer or inhibitor compound; (vi) comparing sweet or umami receptor activity in the test cell after step (iv) and step(v) and if (iv) is respectively greater or lesser than (v) identifying said compound as a putative sweet or umami enhancer or inhibitor. (Again putative sweet or umami enhancer or inhibitor activity can be confirmed, e.g., in suitable taste tests or therapeutic screens.).

In the foregoing assay, the sweet or umami receptor is preferably human or rodent or a chimera thereof e.g., comprising the extracellular domain of a first T1R and the transmembrane domain of a second T1R, and/or the ligand binding regions. Such test cells are preferably endogenous taste cells or mammalian cells such as HEK-293, CHO, COS or BHK cells or frog oocytes, the calcium or voltage specific dye or label is preferably a fluorimetric calcium indicator compound or a voltage specific compound such as Fluo-3, Fluo-4, Fura-2, indo-1, Quin-2, Oregon green, Calcium green 2 or a calcium sensitive protein such as luciferase, aequorin or apo-aequorin, or specifically Fluo3AM, and the detection of changes in sweet or umami receptor activity is detected based on changes in fluorescence or cell voltage and moreover preferably is effected automatically such as by use of a fluorimetric imaging plate reader (FLIPR) or by use of a voltage imaging plate reader (VIPR). In such assays the sweet or umami compound may be a natural or synthetic compound that elicits sweet or umami taste, such as a synthetic sweetener, e.g., aspartame, Sucralose, saccharin, monellin and the like or a natural sweet compound such as glucose, sucrose, lactose, fructose and the like.

It is another object of the invention to provide compositions containing the identified enhancer or inhibitor compounds or derivatives thereof which function as enhancers or inhibitors, and optionally the compound that it enhances, such as food, beverage, and medicament compositions and potentially as therapeutics.

It is another object of the invention to identify the specific site or residues on the sweet or umami taste receptor that the enhancer compound interacts.

### BRIEF DESCRIPTION OF THE FIGURES

FIG. 1 Novel assay conditions and identification of a sweet receptor enhancer. HEK293 cells expressing the human Sweet receptor and G□15 were loaded with Fluo3AM. Cells were stimulated with 50 uM S2423 (arrow) and incubated for 7.5 minutes (pre-incubation step). A suboptimal concentration of Sucralose (Trace #1) was then added to the wells of the plate. The well that had been pre-incubated with S2423 showed a much greater response (Trace #3) that corresponded to about ˜70% of the maximum receptor response obtained with a saturating concentration of Sucralose (Trace #2).

FIG. 2 Effect of increasing concentrations of S2423. HEK293 cells expressing the human Sweet receptor and G□15 were loaded with Fluo3AM and tested using a stimulation protocol similar to the one described in FIG. 1. In this case, however, cells were pre-incubated with increasing concentrations of S2423 and then stimulated with D-PBS or a low concentration of Sucralose. At each concentrations tested, S2423 becomes only active in the presence of Sucralose, one of the hallmarks for a receptor enhancer.

FIG. 3 Effect of S2423 on Sucralose dose-response in the assay. HEK293 cells expressing the human Sweet receptor and G□15 were loaded with Fluo3AM and tested using a stimulation protocol similar to the one described in FIG. 1. In this case, however, cells were pre-incubated with a fixed concentration of S2423 (50 uM or 25 uM final) and then stimulated with increasing concentrations of Sucralose. S2423 increases the potency of Sucralose in the assay by ˜4-5 fold (from 53 uM to 11 uM).

FIG. 4A-F Effect of S2423 on sweeteners and GPCR agonists dose-response in the assay. HEK293 cells expressing the human Sweet receptor and Galpha15 were loaded with Fluo3AM and tested using a stimulation protocol similar to the one described in FIG. 1. In this case, however, cells were pre-incubated with a fixed concentration of S2423 (50 uM or 25 uM final) and then stimulated with increasing concentrations of Sucralose, D-Fructose, Sucrose, Aspartame, Carbachol and Isoproterenol. S2423 specifically increases the potency of Sucralose in the assay and shows no effect on the other sweeteners and un-related GPCR agonists.

FIG. 5A-B S2423 requires pre-incubation for enhancement. HEK293 cells expressing the human Sweet receptor and G□15 were loaded with Fluo3AM. Left panel describes results obtained when performing an experiment identical to the one described in FIG. 3. The right panel describes results obtained when S2423 was added simultaneously with the sweetener (instead of using a pre-incubation protocol). S2423 enhancement can only be picked up upon pre-incubation.

### DETAILED DESCRIPTION OF THE INVENTION

Prior to specifically describing the invention, the following definitions are provided.

The term “T1R” family includes polymorphic variants, alleles, mutants, and homologs that: (1) have about 30-40% amino acid sequence identity, more specifically about 40, 50, 60, 70, 75, 80, 85, 90, 95, 96, 97, 98, or 99% amino acid sequence identity to the T1Rs disclosed infra, and in the patent applications incorporated, by reference herein over a window of about 25 amino acids, optimally 50-100 amino acids; (2) specifically bind to antibodies raised against an immunogen comprising an amino acid sequence selected from the group consisting of the T1R sequences disclosed infra, and conservatively modified variants thereof; (3) specifically hybridize (with a size of at least about 100, optionally at least about 500-1000 nucleotides) under stringent hybridization conditions to a sequence selected from the group consisting of the T1R DNA sequences disclosed infra, and conservatively modified variants thereof; (4) comprise a sequence at least about 40% identical to an amino acid sequence selected from the group consisting of the T1R amino acid sequences disclosed infra or (5) are amplified by primers that specifically hybridize under stringent hybridization conditions to the described T1R sequences. These sequences and functional chimeras, variants and the domains thereof are also disclosed in the Senomyx patent applications incorporated by reference herein.

In particular, these “T1R's” include taste receptor GPCRs referred to as hT1R1, hT1R2, hT1R3, rT1R1, rT1R2, rT1R3, mT1R1, mT1R2, and mT1R3 having the nucleic acid sequences and amino acid sequences provided in this application, and variants, alleles, mutants, orthologs and chimeras thereof which specifically bind and/or respond to sweet or umami ligands including activators, inhibitors and enhancers. Also, T1R polypeptides include chimeric sequences derived from portions of a particular T1R polypeptide such a T1R1, T1R2 or T1R3 of different species or by combining portions of different T1Rs wherein such chimeric T1R sequences combine to produce a functional sweet or umami taste receptor. For example chimeric T1Rs according to the invention may comprise the extracellular region of one T1R, i.e., T1R1 or T1R2 and the transmembrane region of another T1R, either T1R1 or T1R2.

Topologically, certain chemosensory GPCRs have an “N-terminal domain;” “extracellular domains,” a “transmembrane domain” comprising seven transmembrane regions, and corresponding cytoplasmic and extracellular loops, “cytoplasmic regions,” and a “C-terminal region” (see, e.g., Hoon et al, Cell, 96:541-51 (1999); Buck & Axel, Cell, 65:175-87 (1991)). These regions can be structurally identified using methods known to those of skill in the art, such as sequence analysis programs that identify hydrophobic and hydrophilic domains (see, e.g., Stryer, Biochemistry, (3rd ed. 1988); see also any of a number of Internet based sequence analysis programs, such as those found at dot.imgen.bcm.tmc.edu). These regions are useful for making chimeric proteins and for in vitro assays of the invention, e.g., ligand binding assays.

“Extracellular domains” therefore refers to the domains of T1R polypeptides that protrude from the cellular membrane and are exposed to the extracellular face of the cell. Such regions would include the “N-terminal domain” that is exposed to the extracellular face of the cell, as well as the extracellular loops of the transmembrane domain that are exposed to the extracellular face of the cell, i.e., the extracellular loops between transmembrane regions 2 and 3, transmembrane regions 4 and 5, and transmembrane regions 6 and 7. The “N-terminal domain” starts at the N-terminus and extends to a region close to the start of the transmembrane region. These extracellular regions are useful for in vitro ligand binding assays, both soluble and solid phase. In addition, transmembrane regions, described below, can also be involved in ligand binding, either in combination with the extracellular region or alone, and are therefore also useful for in vitro ligand binding assays.

“Transmembrane domain,” which comprises the seven transmembrane “regions,” refers to the domain of T1R polypeptides that lies within the plasma membrane, and may also include the corresponding cytoplasmic (intracellular) and extracellular loops, also referred to as transmembrane “regions.” The seven transmembrane regions and extracellular and cytoplasmic loops can be identified using standard methods, as described in Kyte & Doolittle, J. Mol. Biol., 157:105-32 (1982)), or in Stryer, supra. The transmembrane domains or regions of human, rat, and murine T1R1, T1R2 and T1R3 are also contained in FIG. 6.

“Cytoplasmic domains” refers to the domains of T1R proteins that face the inside of the cell, e.g., the “C-terminal domain” and the intracellular loops of the transmembrane domain, e.g., the intracellular loops between transmembrane regions 1 and 2, transmembrane regions 3 and 4, and transmembrane regions 5 and 6. “C-terminal domain” refers to the region that spans from the end of the last transmembrane region to the C-terminus of the protein, and which is normally located within the cytoplasm.

The term “7-transmembrane receptor” means a polypeptide belonging to a superfamily of transmembrane proteins that have seven regions that span the plasma membrane seven times (thus, the seven regions are called “transmembrane” or “TM” domains TM I to TM VII).

The term “ligand-binding region” refers to sequences derived from a chemosensory or taste receptor that substantially incorporates transmembrane domains II to VII (TM II to VII). The region may be capable of binding a ligand, and more particularly, a taste eliciting compound. These regions are also described in detail in U.S. Ser. No. 10/569,870 incorporated by reference in its entirety herein.

The term “plasma membrane translocation domain” or simply “translocation domain” means a polypeptide domain that when incorporated into the amino terminus of a polypeptide coding sequence, can with great efficiency “chaperone” or “translocate” the hybrid (“fusion”) protein to the cell plasma membrane. An exemplary “translocation domain” is derived from the amino terminus of the human rhodopsin receptor polypeptide, a 7-transmembrane receptor. Another translocation domain is known is the bovine rhodopsin sequence and is also useful for facilitating translocation. Rhodopsin derived sequences are particularly efficient in translocating 7-transmembrane fusion proteins to the plasma membrane.

The phrase “functional effects” or “activity” in the context of the disclosed assays for testing compounds that modulate (enhance) T1R family member mediated taste transduction such as sweet or umami receptor functional effects or activity includes the determination of any parameter that is indirectly or directly under the influence of the particular sweet or umami taste receptor, e.g., functional, physical and chemical effects. It includes ligand binding, changes in ion flux, membrane potential, current flow, transcription, G protein binding, GPCR phosphorylation or dephosphorylation, signal transduction, receptor-ligand interactions, second messenger concentrations (e.g., cAMP, cGMP, IP3, or intracellular Ca²⁺), in vitro, in vivo, and ex vivo and also includes other physiologic effects such increases or decreases of neurotransmitter or hormone release.

By “determining the functional effect” or receptor “activity” is meant assays for a compound that increases or decreases a parameter that is indirectly or directly under the influence of a T1R family member, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, oocyte T1R gene expression; tissue culture cell T1R expression; transcriptional activation of T1R genes; ligand binding assays; voltage, membrane potential and conductance changes; ion flux assays; changes in intracellular second messengers such as cAMP, cGMP, and inositol triphosphate (IP3); changes in intracellular calcium levels; neurotransmitter release, and the like.

“Inhibitors,” “activators,” and “modulators” of T1R proteins receptors are used interchangeably to refer to inhibitory, activating, or modulating molecules identified using in vitro and in vivo assays for taste transduction, e.g., ligands, agonists, antagonists, and their homologs and mimetics. Inhibitors are compounds that, e.g., bind to, partially or totally block stimulation, decrease, prevent, delay activation, inactivate, desensitize, or down regulate taste transduction, e.g., antagonists. Activators are compounds that, e.g., bind to, stimulate, increase, open, activate, facilitate, enhance activation, sensitize, or up regulate taste transduction and/or other functions of T1Rs such as glucose, insulin and fat transport, metabolism, and/or absorption; the release of transporters and satiety peptides, weight gain, diabetes, obesity, and the like, e.g., agonists or antagonists. Modulators include compounds that, e.g., alter the interaction of a receptor with extracellular proteins that bind activators or inhibitor; G Proteins; kinases (e.g., homologs of rhodopsin kinase and beta adrenergic receptor kinases that are involved in deactivation and desensitization of a receptor); and arresting, which also deactivate and desensitize receptors. Modulators include genetically modified versions of T1R family members, e.g., with altered activity, as well as naturally occurring and synthetic ligands, antagonists, agonists, small chemical molecules and the like. In the present invention this includes in particular sweet ligands (agonists or antagonists), umami ligands (agonists and antagonists), sweet enhancers or sweet inhibitors and umami enhancers and inhibitors. As noted, these compounds may modulate taste and/or other T1R associated metabolic functions and associated diseases as above-described.

“Enhancer” herein refers to a compound that modulates (increases) the activation of a particular receptor, preferably the sweet (T1R2/T1R3) receptor or the umami (T1R1/T1R3) taste receptor but which compound by itself does not result in the activation of the particular receptor. Herein such enhancers will enhance the activation of the sweet or umami receptor by a sweet or umami compound. Typically the “enhancer” will be specific to a particular sweet or umami compound, i.e., it will not enhance the activation of the sweet or umami receptor by sweet or umami compounds other than the particular sweet or umami compound or compounds closely related thereto. By contrast an “inhibitor” herein refers to a compound that modulates (reduces) the activation of a particular receptor, e.g., the sweet (T1R2/T1R3) receptor or the umami (T1R1/T1R3) taste receptor but which compound by itself does not affect in the activation of the particular receptor. Herein such inhibitors will e.g., inhibit the activation of the sweet or umami receptor by a sweet or umami or another compound. Typically the “inhibitor” will be specific to a particular sweet or umami compound.

“Putative enhancer” herein more specifically includes compounds identified as a potential enhancer using assays which are described herein but which enhancer activity has not yet been confirmed in vivo, e.g., in suitable taste tests. “Putative inhibitor” herein more specifically includes compounds identified as potential inhibitors using assays which are described herein but which inhibitor activity has not yet been confirmed in vivo, e.g., in suitable taste tests and/or tests evaluating the effect thereof in vivo in various gastrointestinal and metabolic functions such as therapeutics for treating conditions such as diabetes, obesity, weight control, fat metabolism, glucose metabolism, insulin metabolism, satiety and/or the release of satiety peptides such as GLP-1.

Assays for inhibitors and activators and enhancers include, e.g., expressing T1R family members in cells or cell membranes, applying putative modulator compounds in the presence or absence of compounds that modulate, e.g., sweet and umami compounds, and then determining the functional effects on taste transduction, as described above. These cells may be recombinant or may comprise endogenous cells which express T1R1, T1R2 and/or T1R3. Samples or assays comprising T1R family members that are treated with a potential activator, inhibitor, or modulator are compared to control samples without the inhibitor, activator, or modulator to examine the extent of modulation. Control samples (untreated with modulators) are assigned a relative T1R activity value of 100%. Inhibition of a T1R is achieved when the T1R activity value relative to the control is about 80%, optionally 50% or 25-0%. Activation of a T1R is achieved when the T1R activity value relative to the control is 110%, optionally 150%, optionally 200-500%, or 1000-3000% higher. In the present invention these assays will use chimeric T1Rs that comprise all or a portion of the extracellular portion of T1R1 or T1R2 and all or a portion of the transmembrane domains of another T1R , i.e., T1R2 or T1R1.

“Preincubating” or “preincubation step” herein generally refers to a step by which a receptor or a composition containing or a cell or cell membrane expressing the receptor is contacted with a compound that is to be screened for its potential efficacy as an enhancer of another sweet or umami compound on the activation of the sweet or umami receptor and incubated for a set time, e.g., from a few seconds, minutes or longer prior to a subsequent step wherein the receptor or composition or cell or cell membrane expressing same is contacted with a suboptimal concentration of a known sweet or umami compound. By way of illustration this preincubation time may be around ½ minute to about 10 minutes, 5-10 minutes, or around 7.5 minutes.

“Suboptimal concentration of sweet or umami compound” herein refers to a concentration of a sweet or umami compound that is sufficient to result in the activation of the sweet or umami receptor but which concentration is lower than the concentration of the sweet or umami compound which results in the maximal activation of the sweet or umami receptor. Therefore, the suboptimal concentration is low enough to facilitate detection of further enhanced activation of the receptor which occurs as a result of an enhancer. This concentration may e.g., be 10-75% of the saturation concentration, or from about 10-5-% of the saturation concentration of from about 10-25% of the saturation concentration. This enhanced activation for example may be detected by use of a calcium fluorimetric imaging assay. Such assays may be effected by use of calcium indicators such as Fura-2, Fluo-3, Fluo-4, indo-1, quin-2, oregon green, calcium green 2 or calcium sensitive proteins such as luciferase, aequorin or apo-equorin.

“Endogenous taste cell” herein refers to any native cell, e.g., a taste bud cell or a gastrointestinal system derived cell that expresses a T1R1, T1R2 and/or T1R3 and potentially another taste signaling molecule such as TRPM5 and gustducin. This includes by way of example cells on the tongue, oral cavity, gastrointestinal tract and associated organs such s the esophagus, stomach, gall bladder, pancreas, liver, small and large intestine. These cells can be identified by known cell identification and separation techniques based on T1R expression e.g., by use of appropriate antibodies and FACS and/or magnetic bead cell selection and isolation procedures. “Potential T1R Therapeutic” includes any compound identified herein as a potential T1R1/T1R3 or T1R2/T1R3 enhancer or inhibitor that has potential based on this enhancer or inhibitor activity for treating confirmed in taste tests) and/or as therapeutics for treating conditions modulated by T1R associated functions such as diabetes, obesity, weight control, fat metabolism, glucose transport, glucose metabolism, insulin metabolism, insulin release, fat metabolism and absorption, satiety and/or the release of satiety peptides such as GLP-1 or glucose transporters such as GLUT2 or SGLT1.

“Saturation concentration” of a sweet or umami compound refers to a concentration of the sweetener or umami compound which is sufficient to elicit a maximal detectable response in the activation of the receptor, e.g., when activation is assayed by calcium fluorimetric imaging or another assay method suitable for detecting the activation of a GPCR.

The terms “purified,” “substantially purified,” and “isolated” as used herein refer to the state of being free of other, dissimilar compounds with which the compound of the invention is normally associated in its natural state. Preferably, “purified,” “substantially purified,” and “isolated” means that the composition comprises at least 0.5%, 1%, 5%, 10%, or 20%, and most preferably at least 50% or 75% of the mass, by weight, of a given sample. In one preferred embodiment, these terms refer to the compound of the invention comprising at least 95% of the mass, by weight, of a given sample. As used herein, the terms “purified,” “substantially purified,” and “isolated”, when referring to a nucleic acid or protein, of nucleic acids or proteins, also refers to a state of purification or concentration different than that which occurs naturally in the mammalian, especially human, body. Any degree of purification or concentration greater than that which occurs naturally in the mammalian, especially human, body, including (1) the purification from other associated structures or compounds or (2) the association with structures or compounds to which it is not normally associated in the mammalian, especially human, body, are within the meaning of “isolated.” The nucleic acid or protein or classes of nucleic acids or proteins, described herein, may be isolated, or otherwise associated with structures or compounds to which they are not normally associated in nature, according to a variety of methods and processes known to those of skill in the art.

As used herein, the term “isolated,” when referring to a nucleic acid or polypeptide refers to a state of purification or concentration different than that which occurs naturally in the mammalian, especially human, body. Any degree of purification or concentration greater than that which occurs naturally in the body, including (1) the purification from other naturally-occurring associated structures or compounds, or (2) the association with structures or compounds to which it is not normally associated in the body are within the meaning of “isolated” as used herein. The nucleic acids or polypeptides described herein may be isolated or otherwise associated with structures or compounds to which they are not normally associated in nature, according to a variety of methods and processed known to those of skill in the art.

As used herein, the terms “amplifying” and “amplification” refer to the use of any suitable amplification methodology for generating or detecting recombinant or naturally expressed nucleic acid, as described in detail, below. For example, the invention provides methods and reagents (e.g., specific oligonucleotide primer pairs) for amplifying (e.g., by polymerase chain reaction, PCR) naturally expressed (e.g., genomic or mRNA) or recombinant (e.g., cDNA) nucleic acids of the invention (e.g., taste eliciting compound-binding sequences of the invention) in vivo or in vitro.

The term “expression vector” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes which contain only the minimum elements needed for transcription of the recombinant nucleic acid.

The term “library” means a preparation that is a mixture of different nucleic acid or poly-peptide molecules, such as the library of recombinant generated sensory, particularly taste receptor ligand-binding regions generated by amplification of nucleic acid with degenerate primer pairs, or an isolated collection of vectors that incorporate the amplified ligand-binding regions, or a mixture of cells each randomly transfected with at least one vector encoding an taste receptor.

The term “nucleic acid” or “nucleic acid sequence” refers to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones.

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating, e.g., sequences in which the third position of one or more selected codons is substituted with mixed-base and/or deoxyinosine residues (Batzer et al., Nucleic Acid Res., 19:5081 (1991); Ohtsuka et al., J. Biol. Chem., 260:2605-08 (1985); Rossolini et al., Mol. Cell. Probes, 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The “translocation domain,” “ligand-binding region”, and T1R receptor compositions described herein also include “analogs,” or “conservative variants” and “mimetics” (“peptidomimetics”) with structures and activity that substantially correspond to the exemplary sequences. Thus, the terms “conservative variant” or “analog” or “mimetic” refer to a polypeptide which has a modified amino acid sequence, such that the change(s) do not substantially alter the polypeptide's (the conservative variant's) structure and/or activity, as defined herein. These include conservatively modified variations of an amino acid sequence, i.e., amino acid substitutions, additions or deletions of those residues that are not critical for protein activity, or substitution of amino acids with residues having similar properties (e.g., acidic, basic, positively or negatively charged, polar or non-polar, etc.) such that the substitutions of even critical amino acids does not substantially alter structure and/or activity.

More particularly, “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein.

For instance, the codons GCA, GCC, GCG and GCU all encode the amino acid alanine. Thus, at every position where an alanine is specified by a codon, the codon can be altered to any of the corresponding codons described without altering the encoded polypeptide.

Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

Conservative substitution tables providing functionally similar amino acids are well known in the art. For example, one exemplary guideline to select conservative substitutions includes (original residue followed by exemplary substitution): ala/gly or ser; arg/lys; asn/gln or his; asp/glu; cys/ser; gln/asn; gly/asp; gly/ala or pro; his/asn or gln; ile/leu or val; leu/ile or val; lys/arg or gln or glu; met/leu or tyr or ile; phe/met or leu or tyr; ser/thr; thr/ser; trp/tyr; tyr/trp or phe; val/ile or leu. An alternative exemplary guideline uses the following six groups, each containing amino acids that are conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (I); 5) Isoleucine (I), Leucine (L), Methionine (M), Valine (V); and 6) Phenylalanine (F), Tyrosine (Y), Tryptophan (W); (see also, e.g., Creighton, Proteins, W. H. Freeman and Company (1984); Schultz and Schimer, Principles of Protein Structure, Springer-Verlag (1979)). One of skill in the art will appreciate that the above-identified substitutions are not the only possible conservative substitutions. For example, for some purposes, one may regard all charged amino acids as conservative substitutions for each other whether they are positive or negative. In addition, individual substitutions, deletions or additions that alter, add or delete a single amino acid or a small percentage of amino acids in an encoded sequence can also be considered “conservatively modified variations.”

The terms “mimetic” and “peptidomimetic” refer to a synthetic chemical compound that has substantially the same structural and/or functional characteristics of the polypeptides, e.g., translocation domains, ligand-binding regions, or chimeric receptors of the invention. The mimetic can be either entirely composed of synthetic, non-natural analogs of amino acids, or may be a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

As with polypeptides of the invention which are conservative variants, routine experimentation will determine whether a mimetic is within the scope of the invention, i.e., that its structure and/or function is not substantially altered. Polypeptide mimetic compositions can contain any combination of non-natural structural components, which are typically from three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. A polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C═O)—CH₂ for —C(.═O)—NH—), aminomethylene (CH₂NH), ethylene, olefin (CH.dbd.CH), ether (CH₂O), thioether (CH₂—S), tetrazole (CN₄), thiazole, retroamide, thioamide, or ester (see, e.g., Spatola, Chemistry and Biochemistry of Amino Acids, Peptides and Proteins, Vol. 7, 267-357, Marcell Dekker, Peptide Backbone Modifications, NY (1983)). A polypeptide can also be characterized as a mimetic by containing all or some non-natural residues in place of naturally occurring amino acid residues; non-natural residues are well described in the scientific and patent literature.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins which can be made detectable, e.g., by incorporating a radiolabel into the peptide or used to detect antibodies specifically reactive with the peptide.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are optionally directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, Similarly, a heterologous protein indicates that the protein comprises two or more subsequences that are not found in the same relationship to each other in nature (e.g., a fusion protein).

A “promoter” is defined as an array of nucleic acid sequences that direct transcription of a nucleic acid. As used herein, a promoter includes necessary nucleic acid sequences near the start site of transcription, such as, in the case of a polymerase 11 type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

As used herein, “recombinant” refers to a polynucleotide synthesized or otherwise manipulated in vitro (e.g., “recombinant polynucleotide”), to methods of using recombinant polynucleotides to produce gene products in cells or other biological systems, or to a polypeptide (“recombinant protein”) encoded by a recombinant polynucleotide. “Recombinant means” also encompass the ligation of nucleic acids having various coding regions or domains or promoter sequences from different sources into an expression cassette or vector for expression of, e.g., inducible or constitutive expression of a fusion protein comprising a translocation domain of the invention and a nucleic acid sequence amplified using a primer of the invention.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Such hybridizations and wash steps can be carried out for, e.g., 1, 2, 5, 10, 15, 30, 60; or more minutes.

Nucleic acids that do not hybridize to each other under stringent conditions are still substantially related if the polypeptides which they encode are substantially related. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such hybridizations and wash steps can be carried out for, e.g., 1, 2, 5, 10, 15, 30, 60, or more minutes. A positive hybridization is at least twice background. Those of ordinary skill will readily recognize that alternative hybridization and wash conditions can be utilized to provide conditions of similar stringency.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-T1R” antibody is an antibody or antibody fragment that specifically binds a polypeptide encoded by a T1R gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or, “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein.

For example, polyclonal antibodies raised to a T1R family member from specific species such as rat, mouse, or human can be selected to obtain only those polyclonal antibodies that are specifically immunoreactive with the T1R polypeptide or an immunogenic portion thereof and not with other proteins, except for orthologs or polymorphic variants and alleles of the T1R polypeptide. This selection may be achieved by subtracting out antibodies that cross-react with T1R molecules from other species or other T1R molecules. Antibodies can also be selected that recognize only T1R GPCR family members but not GPCRs from other families. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual, (1988), for a description of immunoassay formats and conditions that can be used to determine specific immunoreactivity). Typically a specific or selective reaction will be at least twice background signal or noise and more typically more than 10 to 100 times background.

The phrase “selectively associates with” refers to the ability of a nucleic acid to “'selectively hybridize” with another as defined above, or the ability of an antibody to if selectively (or specifically) bind to a protein, as defined above.

The term “expression vector” refers to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression “cassettes which contain only the minimum elements needed for transcription of the recombinant nucleic acid.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa, HEK-293, and the like, e.g., cultured cells, explants, and cells in vivo.

As described in more detail infra and as exemplified in the working examples, the subject invention relates to the discovery that enhancers or inhibitors of T1R taste receptors, i.e., the T1R2/T1R3 sweet receptor or the T1R1/T1R3 umami taste receptor can be identified more efficiently during screening assays, preferably high throughput compound screening assays, if the sweet or umami taste receptor is preincubated with a potential enhancer compound prior to a subsequent step wherein the sweet or umami taste receptor is contacted with a known activator of the sweet or umami taste receptor, i.e., a natural or synthetic sweet or umami tastant. It has been surprisingly discovered that by contrast if the potential modulator and the known activator of the T1R taste receptor are contacted with the T1R receptor in combination that such assays may not detect the enhancement or inhibitory effect of the potential enhancer or inhibitor on the known activator of the T1R receptor.

While it is not known why this occurs, it is hypothesized by the inventors that the potential enhancer or inhibitor may only after a sufficient time of contact with the T1R receptor, e.g. the sweet or umami receptor, induce some change in the conformational structure of the T1R receptor that does not itself induce activation but which change facilitates or inhibits the ability of the subsequently added activator to thereupon activate the T1R receptor, perhaps by enhancing or inhibiting the extent or stability of activator binding or by altering the binding of the activator (e.g. particular T1R residues that interact with the activator) to the T1R receptor.

Therefore, the present assays can detect T1R enhancers or inhibitors which are undetected using T1R enhancer or inhibitor assays that do not include such a preincubation step. Therefore, these improved assays may be used to more efficiently screen compound libraries for compounds that enhance or inhibit the sweet or umami taste elicited by other sweet or umami tasting compounds.

The subject enhancers and inhibitors are useful as flavor additives to foods, beverages, medicaments and the like. For example, they may be useful in producing foods, beverages, and medicaments with a desired level of sweet or umami taste that contain a reduced amount of a particular sweet or umami compound such as a natural sweetener or synthetic sweetener or umami tastant. This is desirable from a dietary health perspective as some sweet tastants may result in undesired increase in calorie content. In addition it is desirable as some sweet and umami tastants may elicit undesired effects in some users such as bitter aftertaste and migraine headache which undesired effects may be alleviated or even eliminated at reduced concentrations. Also, these compounds can be used as therapeutics e.g., for regulating, treating, and/or preventing conditions and biological functions such as obesity, diabetes, glucose transport, absorption, fat metabolism, fat absorption, insulin release, insulin metabolism, weight control, satiety, satiety peptide release (e.g., GLP-1) and/or glucose transporter release (e.g. GLUT2 or SGLT1)

Also, these newly discovered enhancer or inhibitor compounds potentially can be used in mapping and functional studies to determine at what residues the enhancer compounds interact with T1R receptor polypeptides and further how they modulate (enhance) the interaction thereof with sweet and umami ligands and their respective taste receptors. Also, these molecules can be used to elucidate the mechanism of the sweet and umami receptors' activation and enhancement or inhibition of activation.

As discussed in detail below, the novel T1R enhancer and inhibitor screening assays of the present invention which include a preincubation step wherein the T1R receptor is contacted with the potential enhancer or inhibitor compound prior to a subsequent step whereby the T1R receptor is contacted with a known activator compound include the specific types of T1R assays which are disclosed in Applicants' earlier T1R related applications including U.S. Ser. No. 09/897,427 filed on Jul. 3, 2001, U.S. Ser. No. 10/179,373 filed on Jun. 26, 2002, and U.S. Ser. No. 10/569,870 filed on Aug. 6, 2004. These patent applications and the references cited therein are incorporated by reference in their entirety herein. Additionally, as discussed below, these assays may be effected using T1R receptors expressed using any of the expression vectors, and cells disclosed herein including endogenous cells that express T1Rs. Other preferred cells for expression include cells typically used in GPCR assays such as HEK-293, CHO, COS, MDK, BHK, monkey L and (frog) oocytes. (In the working examples the subject assays are exemplified using HEK-293 cells which express the human sweet hT1R2/hT1R3 taste receptor)

In functional cell based assays such as those discussed below the T1R receptor will preferably be expressed in association with a suitable G protein such as a promiscuous G protein such as Galpha15, Galpha16, transducin, gustducin, a Gq protein, a Gi protein or a chimeric G protein such as a chimeric protein derived from Galpha16 and gustducin or transducin. This may include as well chimeric G proteins derived from Galpha15 and/or transducin or gustducin.

Also, it should be understood that while the application exemplifies sweet or umami receptors comprised of specific T1R1, T1R2 and T1R3 nucleic acid and protein sequences that the invention further contemplates variants thereof, e.g., nucleic acid sequences and polypeptides that poses at least 80% sequence identity therewith, more preferably at least 90% sequence identity therewith, and more typically from 95, 96, 97, 98, or 99% sequence identity therewith. Similarly, these T1R sequences may be expressed in association with wild-type or variant T1R sequences, i.e., variants which possess at least 80% sequence identity to human or rodent T1R3, more typically at least 90% sequence identity therewith, and even more typically at lest 95, 96, 97, 98 or 99% sequence identity therewith.

The taste modulatory effect of the putative umami and sweet enhancers and inhibitors identified using the subject chimeric taste receptors can be confirmed in human or animal taste tests. For example it will be confirmed that they modulate sweet or umami taste only or in association with other compounds (sweet compound or umami tasting compound). These compounds may be used as flavor additives in various compositions including foods, beverages, medicaments and cosmetics. The therapeutic efficacy an be confirmed in appropriate in vitro and in vivo screening assays such as those using transgenic rodents

Preferably, these assays will utilize a test cell that expresses a DNA encoding an hT1R having one of the amino acid sequences identified infra. However, it is anticipated that fragments, orthologs, variants or chimeras of these receptor polypeptides which retain the functional properties of these chimeric sweet-umami or umami-sweet taste receptors, i.e., respond to some sweet or umami compounds or enhancers thereof compounds, will also be useful in these assays. Examples of such variants include splice variants, single nucleotide polymorphisms, allelic variants, and mutations produced by recombinant or chemical means, or naturally occurring. Means for isolation and expression of T1Rs, which are used in the assays of the present invention and assays which are contemplated for use in the present invention to identify compounds that inhibit activation of these receptors, are set forth below.

### Isolation and Expression of T1Rs

Isolation and expression of the T1Rs, or fragments or variants thereof, of the invention can be effected by well-established cloning procedures using probes or primers constructed based on the T1R nucleic acids sequences disclosed in the application. Related T1R sequences may also be identified from human or other species genomic databases using the sequences disclosed herein and known computer-based search technologies, e.g., BLAST sequence searching. In a particular embodiment, the pseudogenes disclosed herein can be used to identify functional alleles or related genes.

Expression vectors can then be used to infect or transfect host cells for the functional expression of these sequences. These genes and vectors can be made and expressed in vitro or in vivo. One of skill will recognize that desired phenotypes for altering and controlling nucleic acid expression can be obtained by modulating the expression or activity of the genes and nucleic acids (e.g., promoters, enhancers and the like) within the vectors of the invention. Any of the known methods described for increasing or decreasing expression or activity can be used. The invention can be practiced in conjunction with any method or protocol known in the art, which are well described in the scientific and patent literature.

Alternatively, these nucleic acids can be synthesized in vitro by well-known chemical synthesis techniques, as described in, e.g., Carruthers, Cold Spring Harbor Symp. Quant. Biol. 47:411-18 (1982); Adams, Am. Chem. Soc., 105:661 (1983); Belousov, Nucleic Acids Res. 25:3440-3444 (1997); Frenkel, Free Radic. Biol. Med. 19:373-380 (1995); Blommers, Biochemistry 33:7886-7896 (1994); Narang, Meth. Enzymol. 68:90 (1979); Brown, Meth. Enzymol. 68:109 (1979); Beaucage, Tetra. Lett. 22:1859 (1981); U.S. Pat. No. 4,458,066. Double-stranded DNA fragments may then be obtained either by synthesizing the complementary strand and annealing the strands together under appropriate conditions, or by adding the complementary strand using DNA polymerase with an appropriate primer sequence.

Techniques for the manipulation of nucleic acids, such as, for example, for generating mutations in sequences, subcloning, labeling probes, sequencing, hybridization and the like are well described in the scientific and patent literature. See, e.g., Sambrook, ed., Molecular Cloning: A Laboratory Manual (2nd ed.), Vols. 1-3, Cold Spring Harbor Laboratory (1989); Ausubel, ed., Current Protocols in Molecular Biology, John Wiley & Sons, Inc., New York (1997); Tijssen, ed., Laboratory Techniques in Biochemistry and Molecular Biology: Hybridization With Nucleic Acid Probes, Part I, Theory and Nucleic Acid Preparation, Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g., fluid or gel precipitin reactions, immunodiffusion, immunoelectrophoresis, radioimmunoassays (R1 As), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

Oligonucleotide primers may be used to amplify nucleic acids encoding a T1R ligand-binding region. The nucleic acids described herein can also be cloned or measured quantitatively using amplification techniques. Amplification methods are also well known in the art, and include, e.g., polymerase chain reaction (PCR) (Innis ed., PCR Protocols, a Guide to Methods and Applications, Academic Press, N.Y. (1990); Innis ed., PCR Strategies, Academic Press, Inc., N.Y. (1995)); ligase chain reaction (LCR) (Wu, Genomics, 4:560 (1989); Landegren, Science, 241:1077 (1988); Barringer, Gene, 89:117 (1990)); transcription amplification (Kwoh, PNAS, 86:1173 (1989)); self-sustained sequence replication (Guatelli, PNAS, 87:1874 (1990)); Q Beta replicase amplification (Smith, J. Clin. Microbiol., 35:1477-91 (1997)); automated Q-beta replicase amplification assay (Burg, Mol. Cell. Probes, 10:257-71 (1996)); and other RNA polymerase mediated techniques (e.g., NASBA, Cangene, Mississauga, Ontario). See also, Berger, Methods Enzymol., 152:307-16 (1987); Sambrook; Ausubel; U.S. Pat. Nos. 4,683,195 and 4,683,202; Sooknanan, Biotechnology, 13:563-64 (1995).

Once amplified, the nucleic acids, either individually or as libraries, may be cloned according to methods known in the art, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” the PCR primer pair. For example, Pst I and Bsp E1 sites were designed into the exemplary primer pairs of the invention. These particular restriction sites have a sequence that, when ligated, are “in-frame” with respect to the 7-membrane receptor “donor” coding sequence into which they are spliced (the ligand-binding region coding sequence is internal to the 7-membrane polypeptide, thus, if it is desired that the construct be translated downstream of a restriction enzyme splice site, out of frame results should be avoided; this may not be necessary if the inserted ligand-binding region comprises substantially most of the transmembrane VII region). The primers can be designed to retain the original sequence of the “donor” 7-membrane receptor. Alternatively, the primers can encode amino acid residues that are conservative substitutions (e.g., hydrophobic for hydrophobic residue, see above discussion) or functionally benign substitutions (e.g., do not prevent plasma membrane insertion, cause cleavage by peptidase, cause abnormal folding of receptor, and the like).

The primer pairs may be designed to selectively amplify ligand-binding regions of T1R proteins. These binding regions may vary for different ligands; thus, what may be a minimal binding region for one ligand, may be too limiting for a second potential ligand. Thus, binding regions of different sizes comprising different domain structures may be amplified; for example, transmembrane (TM) domains II through VII, III through VII, III through VI or II through VI, or variations thereof (e.g., only a subsequence of a particular domain, mixing the order of the domains, and the like), of a 7-transmembrane T1R.

As domain structures and sequence of many 7-membrane T1R proteins are known, the skilled artisan can readily select domain-flanking and internal domain sequences as model sequences to design degenerate amplification primer pairs. For example, a nucleic acid sequence encoding domain regions II through VII can be generated by PCR amplification using a primer pair. To amplify a nucleic acid comprising transmembrane domain I (TM I) sequence, a degenerate primer can be designed from a nucleic acid that encodes the amino acid sequence of the T1R family consensus sequence 1 described above. Such a degenerate primer can be used to generate a binding region incorporating TM I through TM III, TM I through TM IV, TM I through TM V, TM I through TM VI or TM I through TM VII). Other degenerate primers can be designed based on the other T1R family consensus sequences provided herein. Such a degenerate primer can be used to generate a binding region incorporating TM III through TM IV, TM III through TM V, TM III through TM VI or TM III through TM VII.

Paradigms to design degenerate primer pairs are well known in the art. For example, a COnsensus-DEgenerate Hybrid Oligonucleotide Primer (CODEHOP) strategy computer program is accessible as http://blocks.fhcrc.org/codehop.html, and is directly linked from the BlockMaker multiple sequence alignment site for hybrid primer prediction beginning with a set of related protein sequences, as known taste receptor ligand-binding regions (see, e.g., Rose, Nucleic Acids Res., 26:1628-35 (1998); Singh, Biotechniques, 24:318-19 (1998)).

Means to synthesize oligonucleotide primer pairs are well known in the art. “Natural” base pairs or synthetic base pairs can be used. For example, use of artificial nucleobases offers a versatile approach to manipulate primer sequence and generate a more complex mixture of amplification products. Various families of artificial nucleobases are capable of assuming multiple hydrogen bonding orientations through internal bond rotations to provide a means for degenerate molecular recognition. Incorporation of these analogs into a single position of a PCR primer allows for generation of a complex library of amplification products. See, e.g., Hoops, Nucleic Acids Res., 25:4866-71 (1997). Nonpolar molecules can also be used to mimic the shape of natural DNA bases. A non-hydrogen-bonding shape mimic for adenine can replicate efficiently and selectively against a nonpolar shape mimic for thymine (see, e.g., Morales, Nat. Struct. Biol., 5:950-54 (1998)). For example, two degenerate bases can be the pyrimidine base 6H, 8H-3,4-dihydropyrimido[4,5-c][1,2]oxazin-7-one or the purine base N6-methoxy-2,6-diaminopurine (see, e.g., Hill, PNAS, 95:4258-63 (1998)). Exemplary degenerate primers of the invention incorporate the nucleobase analog 5′-Dimethoxytrityl-N-benzoyl-2′-deoxy-Cytidine,3′-[(2-cyanoethyl)-(N,N-diisopropyl)]-phosphoramidite (the term “P” in the sequences, see above). This pyrimidine analog hydrogen bonds with purines, including A and G residues.

Polymorphic variants, alleles, and interspecies homologs that are substantially identical to a taste receptor disclosed herein can be isolated using the nucleic acid probes described above. Alternatively, expression libraries can be used to clone T1R polypeptides and polymorphic variants, alleles, and interspecies homologs thereof, by detecting expressed homologs immunologically with antisera or purified antibodies made against a T1R polypeptide, which also recognize and selectively bind to the T1R homolog.

Nucleic acids that encode ligand-binding regions of taste receptors may be generated by amplification (e.g., PCR) of appropriate nucleic acid sequences using appropriate (perfect or degenerate) primer pairs. The amplified nucleic acid can be genomic DNA from any cell or tissue or mRNA or cDNA derived from taste receptor-expressing cells.

In one embodiment, hybrid protein-coding sequences comprising nucleic acids encoding chimeric or native T1Rs fused to a translocation sequences may be constructed. Also provided are hybrid T1Rs comprising the translocation motifs and taste eliciting compound-binding regions of other families of chemosensory receptors, particularly taste receptors. These nucleic acid sequences can be operably linked to transcriptional or translational control elements, e.g., transcription and translation initiation sequences, promoters and enhancers, transcription and translation terminators, polyadenylation sequences, and other sequences useful for transcribing DNA into RNA. In construction of recombinant expression cassettes, vectors, and transgenics, a promoter fragment can be employed to direct expression of the desired nucleic acid in all desired cells or tissues.

In another embodiment, fusion proteins may include C-terminal or N-terminal translocation sequences. Further, fusion proteins can comprise additional elements, e.g., for protein detection, purification, or other applications. Detection and purification facilitating domains include, e.g., metal chelating peptides such as polyhistidine tracts, histidine-tryptophan modules, or other domains that allow purification on immobilized metals; maltose binding protein; protein A domains that allow purification on immobilized immunoglobulin; or the domain utilized in the FLAGS extension/affinity purification system (Immunex Corp, Seattle Wash.).

The inclusion of a cleavable linker sequences such as Factor Xa (see, e.g., Ottavi, Biochimie, 80:289-93 (1998)), subtilisin protease recognition motif (see, e.g., Polyak, Protein Eng., 10:615-19 (1997)); enterokinase (Invitrogen, San Diego, Calif.), and the like, between the translocation domain (for efficient plasma membrane expression) and the rest of the newly translated polypeptide may be useful to facilitate purification. For example, one construct can include a polypeptide encoding a nucleic acid sequence linked to six histidine residues followed by a thioredoxin, an enterokinase cleavage site (see, e.g., Williams, Biochemistry, 34:1787-97 (1995)), and an C-terminal translocation domain. The histidine residues facilitate detection and purification while the enterokinase cleavage site provides a means for purifying the desired protein(s) from the remainder of the fusion protein. Technology pertaining to vectors encoding fusion proteins and application of fusion proteins are well described in the scientific and patent literature (see, e.g., Kroll, DNA Cell. Biol,. 12:441-53 (1993)).

Expression vectors, either as individual expression vectors or as libraries of expression vectors, comprising the ligand-binding region encoding sequences may be introduced into a genome or into the cytoplasm or a nucleus of a cell and expressed by a variety of conventional techniques, well described in the scientific and patent literature. See, e.g., Roberts, Nature, 328:731 (1987); Berger supra; Schneider, Protein Exper. Purif., 6435:10 (1995); Sambrook; Tijssen; Ausubel. Product information from manufacturers of biological reagents and experimental equipment also provide information regarding known biological methods. The vectors can be isolated from natural sources, obtained from such sources as ATCC or GenBank libraries, or prepared by synthetic or recombinant methods.

The nucleic acids can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required. For example, the marker may encode antibiotic resistance (e.g., chloramphenicol, kanamycin, G418, bleomycin, hygromycin) or herbicide resistance (e.g., chlorosulfuron or Basta) to permit selection of those cells transformed with the desired DNA sequences (see, e.g., Blondelet-Rouault, Gene, 190:315-17 (1997); Aubrecht, J. Pharmacol. Exp. Ther., 281:992-97 (1997)). Because selectable marker genes conferring resistance to substrates like neomycin or hygromycin can only be utilized in tissue culture, chemoresistance genes are also used as selectable markers in vitro and in vivo.

A chimeric nucleic acid sequence may encode a T1R ligand-binding region within any 7-transmembrane polypeptide. Because 7-transmembrane receptor polypeptides have similar primary sequences and secondary and tertiary structures, structural domains (e.g., extracellular domain, TM domains, cytoplasmic domain, etc.) can be readily identified by sequence analysis. For example, homology modeling, Fourier analysis and helical periodicity detection can identify and characterize the seven domains with a 7-transmembrane receptor sequence. Fast Fourier Transform (FFT) algorithms can be used to assess the dominant periods that characterize profiles of the hydrophobicity and variability of analyzed sequences. Periodicity detection enhancement and alpha helical periodicity index can be done as by, e.g., Donnelly, Protein Sci., 2:55-70 (1993). Other alignment and modeling algorithms are well known in the art (see, e.g., Peitsch, Receptors Channels, 4:161-64 (1996); Kyte & Doolittle, J. Md. Biol., 157:105-32 (1982); and Cronet, Protein Eng., 6:59-64 (1993).

The present invention also includes not only the nucleic acid molecules and polypeptides having the specified native T1R nucleic and amino acid sequences, but also fragments thereof, particularly fragments of, e.g., 40, 60, 80, 100, 150, 200, or 250 nucleotides, or more, as well as polypeptide fragments of, e.g., 10, 20, 30, 50, 70, 100, or 150 amino acids, or more. Optionally, the nucleic acid fragments can encode an antigenic polypeptide that is capable of binding to an antibody raised against a T1R family member. Further, a protein fragment of the invention can optionally be an antigenic fragment that is capable of binding to an antibody raised against a T1R family member.

Also contemplated are chimeric proteins, comprising at least 10, 20, 30, 50, 70, 100, or 150 amino acids, or more, of one of at least one of the T1R polypeptides described herein, coupled to additional amino acids representing all or part of another GPCR, preferably a member of the 7 transmembrane superfamily. These chimeras can be made from the instant receptors and another GPCR, or they can be made by combining two or more of the present receptors. In one embodiment, one portion of the chimera corresponds to, or is derived from the transmembrane domain of a T1R polypeptide of the invention. In another embodiment, one portion of the chimera corresponds to, or is derived from the one or more of the transmembrane regions of a T1R polypeptide described herein, and the remaining portion or portions can come from another GPCR. Chimeric receptors are well known in the art, and the techniques for creating them and the selection and boundaries of domains or fragments of G Protein-Coupled Receptors for incorporation therein are also well known. Thus, this knowledge of those skilled in the art can readily be used to create such chimeric receptors. The use of such chimeric receptors can provide, for example, a taste selectivity characteristic of one of the receptors specifically disclosed herein, coupled with the signal transduction characteristics of another receptor, such as a well known receptor used in prior art assay systems.

For example, a region such as a ligand-binding region, an extracellular domain, a transmembrane domain, a transmembrane domain, a cytoplasmic domain, an N-terminal domain, a C-terminal domain, or any combination thereof, can be covalently linked to a heterologous protein. For instance, a T1R transmembrane region can be linked to a heterologous GPCR transmembrane domain, or a heterologous GPCR extracellular domain can be linked to a T1R transmembrane region. Other heterologous proteins of choice can include, e.g., green fluorescent protein, .beta.-galactosidase polypeptides, glutamate receptor, and the rhodopsin polypeptides, e.g., N-terminal fragments of rhodopsin e.g., bovine rhodopsin.

It is also within the scope of the invention to use different host cells for expressing the T1Rs, fragments, or variants of the invention. To obtain high levels of expression of a cloned gene or nucleic acid, such as cDNAs encoding the T1Rs, fragments, or variants of the invention, one of skill typically subclones the nucleic acid sequence of interest into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook et al. Preferably, eukaryotic expression systems are used to express the subject hT1R receptor.

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al.) It is only necessary that the particular genetic engineering procedure used be capable of successfully introducing at lest one nucleic acid molecule into the host cell capable of expressing the T1R, fragment, or variant of interest.

After the expression vector is introduced into the cells, the transfected cells are cultured under conditions favoring expression of the receptor, fragment, or variant of interest, which is then recovered from the culture using standard techniques. Examples of such techniques are well known in the art. See, e.g., WO 00/06593, which is incorporated by reference in a manner consistent with this disclosure.

### Suitable Assays for Detection of T1R Binding Compounds

Methods and compositions for determining whether a test compound specifically binds to a T1R polypeptide of the invention, both in vitro and in vivo are described below. Many aspects of cell physiology can be monitored to assess the effect of ligand-binding to a naturally occurring or chimeric T1Rs. These assays may be performed on intact cells expressing a T1R polypeptide, on permeabilized cells, or on membrane fractions produced by standard methods.

Taste receptors bind taste eliciting compounds and initiate the transduction of chemical stimuli into electrical signals. An activated or inhibited G protein will in turn alter the properties of target enzymes, channels, and other effector proteins. Some examples are the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

The subject T1R1/T1R3 or T1R2/T1R3 receptor polypeptides used in the present assays will typically comprise native T1R polypeptide or fragments or variants thereof that retain the same ligand binding properties as the native sweet or umami receptor polypeptides.

Alternatively, the receptor may comprise chimeric T1R proteins or polypeptides such as disclosed supra and exemplified in Applicant's earlier application U.S. Ser. No. (TO BE ADDED)

Generally, the amino acid sequence identity will be at least 30% preferably 30-40%, more specifically 50-60, 70%, 75%, 80%, 85%, 90%, 95%, 96%, 97%, 98%, or 99% to the particular native T1R. Optionally, the T1R proteins or polypeptides of the assays can comprise a region of a T1R polypeptide, such as an extracellular domain, transmembrane region, cytoplasmic domain, ligand-binding domain, and the like. Optionally, the T1R polypeptide, or a portion thereof, can be covalently linked to a heterologous protein to create a chimeric protein used in the assays described herein.

Modulators of T1R activity may be tested using T1R proteins or polypeptides as described above, either recombinant or naturally occurring. The T1R proteins or polypeptides can be isolated, expressed in a cell, expressed in a membrane derived from a cell, expressed in tissue or in an animal, either recombinant or naturally occurring. For example, tongue slices, dissociated cells from a tongue, transformed cells, or membranes can be used. Modulation can be tested using one of the in vitro or in vivo assays described herein.

As described above the invention the present invention provides novel and improved assay methods for identifying enhancers of the sweet or umami taste receptor. While the subject assays are exemplified in the context of sweet enhancer assays and the identification of a novel sweet enhancer compound it is envisioned that the same improved assay methods will be useful in identifying umami enhancers. The inventive novel cell based assay conditions preferably use the human sweet receptor (T1R2 and T1R3) or the human umami (T1R1 and T1R3) expressed in HEK-293 cells, for the identification of hT1R2/hT1R3 enhancers (modulators) or hT1R1/hT1R3 enhancers (modulators). In such assays the T1R2/T1R3 or T1R1/T1R3 taste receptor or cells expressing same will be preincubated with a putative enhancer compound for a specific time, generally from about ½ to around 60 minutes or more typically for about 5-10 minutes prior to the taste receptor being contacted with a known sweet or umami compound that activates the taste receptor. As noted above, it has been surprisingly been discovered that the use of such a preincubation step is important in identifying some enhancer or inhibitor compounds as the enhancer or inhibitor may not be identified in otherwise identical assays wherein the receptor is exposed to a combination of the same concentration of the sweet or umami compound and the same enhancer or inhibitor compound or even increased enhancer compound concentration but in the absence of a preincubation step. Again, while it is not known why this occurs it is theorized that the preincubation may induce some change in the conformation of the sweet or umami taste receptor which does not induce receptor activation but which conformational change may enhance or alter the subsequent binding of the known activator to the particular T1R taste receptor. (The manner by which the enhancer or inhibitor compound modulates the activation of the receptor by the known umami or sweet activator ligand potentially may be elucidated in mapping or other functional studies).

In the subject assays the receptor or cells expressing same will be placed in a suitable environment that facilitates the receptor or cells to be used in the particular assay. For instance, in the exemplified calcium imaging assays cells which express hT1R2/hT1R3-HEK293 G□₁₅ cells were seeded in 384-well-clear bottom plates (Fisher) at a density of ˜50,000 cells/well and grown overnight at 37 degrees C. However, it is anticipated that these cell density conditions and/or cell culture devices potentially may be altered.

On the day of the experiment, cells were loaded with a commercially available calcium indicator dye Fluo3AM (2 uM) (Invitrogen) in D-PBS (Invitrogen) using a Multidrop (Titertek). (Again while Fluo3AM is exemplified it is anticipated that other known and available calcium indicators can be substituted therefore). Cells were incubated for 1 hour at room temperature and excess dye was washed out with D-PBS using an EMBLA cell washer (Molecular Devices), leaving a residual volume of 25 □l/well. After 30 minutes of rest time at room temperature, Fluo3AM-loaded cell plate, a compound plate and a sweetener plate were loaded into a Fluorometric Imaging Plate Reader (FLIPR, Molecular Devices). 384-well compound plate and the sweetener plate were prepared at 3× final concentration in D-PBS. Imaging was initiated with the acquisition of the baseline fluorescence for a period of 7 to 10 seconds, and then cells were stimulated on line with addition of 25 □l/well of the compound plate (pre-incubation step). (Again while the foregoing suboptimal concentration of activator compound was used it is anticipated that these concentrations may be altered e.g., if another activator compound is used or if the assay conditions are otherwise modified.)

In the exemplified assays the cells were preincubated for 7.5 minutes with the compound prior a last stimulation with the sweetener plate. (While this preincubation time has been found to be optimal under the disclosed assay conditions, it is anticipated that this preincubation period potentially may be altered, e.g. if another type of assay is used to assay T1R receptor activity or if the assay conditions are altered in another manner).

As shown in the Figures and the examples infra, this protocol resulted in the discovery of a novel modulator, S2423, which enhances the Sucralose potency on the sweet receptor by about 4 to 5 fold at 25 uM. S2423 is a specific enhancer as it does not enhance the effect of other sweeteners such as D-Fructose, Sucrose, Aspartame and also does not enhance the effects of two other un-related GPCR agonists isoproterenol and carbachol. It is anticipated that the same or similar protocols including the preincubation step will result in the identification of other sweet or umami enhancer compounds. (As demonstrated in experiments discussed in the examples the novel pre-incubation step is important in order to detect the enhancement effect of S2423 as co-stimulation of the cells with sweetener and S2423 does not reveal similar magnitude of enhancement. These results are unexpected and illustrate that the invention provides novel assays which should result in the identification of T1R receptor enhancers not identifiable by other methods.

Detection of T1R Modulators (Enhancers) According to the Invention

The present invention relates to the novel discovery that binding or functional assays designed to identify T1R enhancers may be improved if the T1R receptor is contacted (preincubated) with a potential enhancer for a requisite period of time, typically from about ½ minute to about an hour and more typically from about 5-10 minutes prior to the receptor being contacted with a known T1R ligand and assayed as to whether the potential enhancer compound enhances T1R ligand binding and/or the T1R activity elicited by the T1R ligand. This discovery is exemplified supra and in the examples in the context of calcium imaging assays. However, it is anticipated that other types of assays may be alternatively used.

Compositions and methods for determining whether a test compound specifically binds to a T1R receptor of the invention, both in vitro and in vivo, are described below. Many aspects of cell physiology can be monitored to assess the effect of ligand binding to a T1R polypeptide of the invention. These assays may be performed on intact cells expressing a chemosensory receptor, on permeabilized cells, or on membrane fractions produced by standard methods or in vitro using de novo synthesized proteins.

In vivo, taste receptors bind to taste modulatory compounds and initiate the transduction of chemical stimuli into electrical signals. An activated or inhibited G protein will in turn alter the properties of target enzymes, channels, and other effector proteins. Some examples are the activation of cGMP phosphodiesterase by transducin in the visual system, adenylate cyclase by the stimulatory G protein, phospholipase C by Gq and other cognate G proteins, and modulation of diverse channels by Gi and other G proteins. Downstream consequences can also be examined such as generation of diacyl glycerol and IP3 by phospholipase C, and in turn, for calcium mobilization by IP3.

Alternatively, the T1R proteins or polypeptides of the assay can be derived from a eukaryotic host cell and can include an amino acid subsequence having amino acid sequence identity to the T1R polypeptides disclosed herein, or fragments or conservatively modified variants thereof. Generally, the amino acid sequence identity will be at least 35 to 50%, or optionally 75%, 85%, 90%, 95%, 96%, 97%, 98%, or 99%. Optionally, the T1R proteins or polypeptides of the assays can comprise a domain of a T1R protein, such as an extracellular domain, transmembrane region, transmembrane domain, cytoplasmic domain, ligand-binding domain, and the like. Further, as described above, the T1R protein or a domain thereof can be covalently linked to a heterologous protein to create a chimeric protein used in the assays described herein.

Modulators of T1R receptor activity are tested using T1R proteins or polypeptides as described above, either recombinant or naturally occurring. The T1R proteins or polypeptides can be isolated, expressed in a cell, expressed in a membrane derived from a cell, expressed in tissue or in an animal, either recombinant or naturally occurring. For example, tongue slices, dissociated cells from a tongue, transformed cells, or membranes can be used. Modulation can be tested using one of the in vitro or in vivo assays described herein.

### 1. In Vitro Binding Assays

Taste transduction can also be examined in vitro with soluble or solid state reactions, using the T1R polypeptides of the invention. In a particular embodiment, T1R ligand-binding domains can be used in vitro in soluble or solid state reactions to assay for ligand binding.

It is possible that the ligand-binding domain may be formed by the N-terminal domain together with additional portions of the extracellular domain, such as the extracellular loops of the transmembrane domain.

In vitro binding assays have been used with other GPCRs, such as the metabotropic glutamate receptors (see, e.g., Han and Hampson, J. Biol. Chem. 274:10008-10013 (1999)). These assays might involve displacing a radioactively or fluorescently labeled ligand, measuring changes in intrinsic fluorescence or changes in proteolytic susceptibility, etc.

Ligand binding to a T1R polypeptide according to the invention can be tested in solution, in a bilayer membrane, optionally attached to a solid phase, in a lipid monolayer, or in vesicles. Binding of a modulator can be tested using, e.g., changes in spectroscopic characteristics (e.g., fluorescence, absorbance, refractive index) hydrodynamic (e.g., shape), chromatographic, or solubility properties.

In a preferred embodiment of the invention, a ^{[35S]}GTPγS binding assay can be used. As described above, upon activation of a GPCR, the G(x subunit of the G protein complex is stimulated to exchange bound GDP for GTP. Ligand-mediated stimulation of G protein exchange activity can be measured in a biochemical assay measuring the binding of added radioactively labeled ^{[35S]}GTPγS to the G protein in the presence of a putative ligand. Typically, membranes containing the chemosensory receptor of interest are mixed with a G protein. Potential inhibitors and/or activators and ^{[35S}]GTPγS are added to the assay, and binding of ^{[35S]}GTPγS to the G protein is measured. Binding can be measured by liquid scintillation counting or by any other means known in the art, including scintillation proximity assays (SPA). In other assays formats, fluorescently labeled GTPγS can be utilized.

### 2. Fluorescence Polarization Assays

In another embodiment, Fluorescence Polarization (“FP”) based assays may be used to detect and monitor T1R ligand binding. Fluorescence polarization is a versatile laboratory technique for measuring equilibrium binding, nucleic acid hybridization, and enzymatic activity. Fluorescence polarization assays are homogeneous in that they do not require a separation step such as centrifugation, filtration, chromatography, precipitation, or electrophoresis. These assays are done in real time, directly in solution and do not require an immobilized phase. Polarization values can be measured repeatedly and after the addition of reagents since measuring the polarization is rapid and does not destroy the sample. Generally, this technique can be used to measure polarization values of fluorophores from low picomolar to micromolar levels. This section describes how fluorescence polarization can be used in a simple and quantitative way to measure the binding of ligands to the T1R polypeptides of the invention.

When a fluorescently labeled molecule is excited with plane polarized light, it emits light that has a degree of polarization that is inversely proportional to its molecular rotation. Large fluorescently labeled molecules remain relatively stationary during the excited state (4 nanoseconds in the case of fluorescein) and the polarization of the light remains relatively constant between excitation and emission. Small fluorescently labeled molecules rotate rapidly during the excited state and the polarization changes significantly between excitation and emission. Therefore, small molecules have low polarization values and large molecules have high polarization values. For example, a single-stranded fluorescein-labeled oligonucleotide has a relatively low polarization value but when it is hybridized to a complementary strand, it has a higher polarization value. When using FP to detect and monitor taste eliciting compound-binding which may activate or inhibit the chemosensory receptors of the invention, fluorescence-labeled taste eliciting compounds or auto-fluorescent taste eliciting compounds may be used.

Fluorescence polarization (P) is defined as:

$$P = \frac{[ {Int}_{par} - {Int}_{perp ]}}{[ {Int}_{par} + {Int}_{perp} ]}$$

Where .Intₚₐᵣ is the intensity of the emission light parallel to the excitation light plane and Intₚₑᵣₚ is the intensity of the emission light perpendicular to the excitation light plane. P, being a ratio of light intensities, is a dimensionless number. For example, the Beacon™ and Beacon 2000™. System may be used in connection with these assays. Such systems typically express polarization in millipolarization units (1 Polarization Unit=1000 mP Units).

The relationship between molecular rotation and size is described by the Perrin equation and the reader is referred to Jolley, M. E. (1991) in Journal of Analytical Toxicology, pp. 236-240 incorporated by reference, which gives a thorough explanation of this equation. Summarily, the Perrin equation states that polarization is directly proportional to the rotational relaxation time, the time that it takes a molecule to rotate through an angle of approximately 68.5°. Rotational relaxation time is related to viscosity (eta.), absolute temperature (T), molecular volume (V), and the gas constant (R) by the following equation: 2(Rotational Relaxation Time)=3 V RT.

The rotational relaxation time is small (≃nanosecond) for small molecules (e.g. fluorescein) and large (≃100 nanoseconds) for large molecules (e.g. immunoglobulins). If viscosity and temperature are held constant, rotational relaxation time, and therefore polarization, is directly related to the molecular volume. Changes in molecular volume may be due to interactions with other molecules, dissociation, polymerization, degradation, hybridization, or conformational changes of the fluorescently labeled molecule. For example, fluorescence polarization has been used to measure enzymatic cleavage of large fluorescein labeled polymers by proteases, DNases, and RNases. It also has been used to measure equilibrium binding for protein/protein interactions, antibody/antigen binding, and protein/DNA binding.

### A. Solid State and Soluble High Throughput Assays

In yet another embodiment, the invention encompasses soluble assays using a T1R polypeptide; or a cell or tissue expressing a T1R polypeptide. In another embodiment, the invention provides solid phase based in vitro assays in a high throughput format, where the T1R polypeptide, or cell or tissue expressing the T1R polypeptide is attached to a solid phase substrate or a taste stimulating compound and contacted with a T1R receptor, and binding detected using an appropriate tag or antibody raised against the T1R receptor.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 1000 to about 1500 different compounds. It is also possible to assay multiple compounds in each plate well. It is possible to assay several different plates per day; assay screens for up to about 6,000-20,000 different compounds is possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule which binds the tag (a tag binder) is fixed to a solid support, and the tagged molecule of interest (e.g., the taste transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc., 85:2149-2154 (1963) (describing solid phase synthesis of, e.g., peptides); Geysen et al., J. Immun. Meth., 102:259-274 (1987) (describing synthesis of solid phase components on pins); Frank & Doring, Tetrahedron, 44:60316040 (1988) (describing synthesis of various peptide sequences on cellulose disks); Fodor et al., Science, 251:767-777 (1991); Sheldon et al., Clinical Chemistry, 39(4):718-719 (1993); and Kozal et al., Nature Medicine, 2(7):753759 (1996) (all describing arrays of biopolymers fixed to solid substrates). Non-chemical approaches for fixing tag binders to substrates include other common methods, such as heat, cross-linking by UV radiation, and the like.

### 3. Cell-Based Assays

In a preferred embodiment of the invention, a T1R protein is expressed in a eukaryotic cell either in unmodified forms or as chimeric, variant or truncated receptors with or preferably without a heterologous, chaperone sequence that facilitates its maturation and targeting through the secretory pathway. Such T1R polypeptides can be expressed in any eukaryotic cell, such as HEK-293 cells. Preferably, the cells comprise a functional G protein, e.g., G_{·α15}, or a chimeric G_{·α16}, gustducin or transducin or a chimeric G protein such as G16gust44 that is capable of coupling the chimeric receptor to an intracellular signaling pathway or to a signaling protein such as phospholipase C. Activation of T1R receptors in such cells can be detected using any standard method, such as by detecting changes in intracellular calcium by detecting FURA-2, FLUO-3, FLUO-4, indo-1, quin-2, dependent fluorescence in the cell. Such a FLUO3AM assay is the basis of the experimental findings presented in this application.

Activated GPCR receptors often are substrates for kinases that phosphorylate the C-terminal tail of the receptor (and possibly other sites as well). Thus, activators will promote the transfer of ³²P from radiolabeled ATP to the receptor, which can be assayed with a scintillation counter. The phosphorylation of the C-terminal tail will promote the binding of arrestin-like proteins and will interfere with the binding of G proteins. For a general review of GPCR signal transduction and methods of assaying signal transduction, see, e.g., Methods in Enzymology, vols. 237 and 238 (1994) and volume 96 (1983); Bourne et al., Nature, 10:349:117-27 (1991); Bourne et al., Nature, 348:125-32 (1990); Pitcheret al., Annu. Rev. Biochem., 67:653-92 (1998).

T1R modulation may be assayed by comparing the response of T1R polypeptides according to the invention pretreated or preincubated with a putative T1R modulator and subsequently treated with a known T1R activator ligand to the response of an untreated control sample or a sample containing a known “positive” control. Such putative T1R modulators (preferably enhancers) can include molecules that either inhibit or activate T1R polypeptide activity. In one embodiment, control samples treated with a compound that activates the T1R are assigned a relative T1R activity value of 100. Inhibition of a T1R polypeptide is achieved when the T1R activity value relative to the control sample is about 90%, optionally 50%, optionally 25-0%. Activation of a T1R polypeptide is achieved when the T1R activity value relative to the control is 110%, optionally 150%, 200-500%, or 1000-2000%.

Changes in ion flux may be assessed by determining changes in ionic polarization (i.e., electrical potential) of the cell or membrane expressing a T1R polypeptide. One means to determine changes in cellular polarization is by measuring changes in current (thereby measuring changes in polarization) with voltage-clamp and patch-clamp techniques (see, e.g., the “cell-attached” mode, the “inside-out” mode, and the “whole cell” mode, e.g., Ackerman et al., New Engl. J Med., 336:1575-1595 (1997)). Whole cell currents are conveniently determined using the standard. Other known assays include: radiolabeled ion flux assays and fluorescence assays using voltage-sensitive dyes (see, e.g., Vestergarrd-Bogind et al., J. Membrane Biol., 88:67-75 (1988); Gonzales & Tsien, Chem. Biol., 4:269-277 (1997); Daniel et al., J. Pharmacol. Meth., 25:185-193 (1991); Holevinsky et al., J. Membrane Biology, 137:59-70 (1994)). Such indicators include Fluo-3, Fluo-4, Fura-2, indo-1, quin-2, oregon green, calcium green 2, and calcium sensitive proteins such as luciferase aequorin and apo-aequorin.

The effects of the test compounds upon the function of the polypeptides can be measured by examining any of the parameters described above. Any suitable physiological change that affects GPCR activity can be used to assess the influence of a test potential enhancer compound on the activation of the T1R receptor polypeptides of this invention by another T1R activator ligand, e.g. a natural or artificial sweetener such as Sucralose or aspartame. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca 2+, IP3, cGMP, or cAMP.

Preferred assays for GPCRs include cells that are loaded with ion or voltage sensitive dyes to report receptor activity. Assays for determining activity of such receptors can also use known agonists and antagonists for other G protein-coupled receptors as controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G protein-coupled receptors, promiscuous G proteins such as G_{α15} and G_{α16} can be used in the assay of choice (Wilkie et al., Proc. Nat'l Acad. Sci., 88:10049-10053 (1991)). Alternatively, other G proteins such as gustducin, transducin and chimeric G proteins such as Gα16gust44 or G16t44 may be used.

Receptor activation initiates subsequent intracellular events, e.g., increases in second messengers. Activation of some G protein-coupled receptors stimulates the formation of inositol triphosphate (IP3) through phospholipase C-mediated hydrolysis of phosphatidylinositol (Berridge & Irvine, Nature, 312:315-21 (1984)). IP3 in turn stimulates the release of intracellular calcium ion stores. Thus, a change in cytoplasmic calcium ion levels, or a change in second messenger levels such as IP3 can be used to assess G protein-coupled receptor function. Cells expressing such G protein-coupled receptors may exhibit increased cytoplasmic calcium levels as a result of contribution from both calcium release from intracellular stores and extracellular calcium entry via plasma membrane ion channels.

In a preferred embodiment, T1R polypeptide activity is measured by expressing T1R gene in a heterologous cell with a promiscuous G protein that links the receptor to a phospholipase C signal transduction pathway (see Offermanns & Simon, J. Biol. Chem., 270:15175-15180 (1995)). Preferably, the cell line is HEK-293 (which does not normally express T1R genes) and the promiscuous G protein is G_{α15} (Offermanns & Simon, supra) or a chimeric G protein such as Gα16gust44. Modulation of taste transduction is assayed by measuring changes in intracellular Ca²⁺ levels, which change in response to modulation of the T1R signal transduction pathway via administration of a molecule that associates with the T1R polypeptide. Changes in Ca²⁺ levels are optionally measured using fluorescent Ca²⁺ indicator dyes and fluorometric imaging using indicators such as afore-mentioned, e.g., Fluo3AM.

In another embodiment, phosphatidyl inositol (PI) hydrolysis can be analyzed according to U.S. Pat. No. 5,436,128, herein incorporated by reference. Briefly, the assay involves labeling of cells with 3H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist, to cpm in the presence of buffer control. Likewise, fold inhibition is determined by calculating the ratio of cpm in the presence of antagonist, to cpm in the presence of buffer control (which may or may not contain an agonist).

Other receptor assays can involve determining the level of intracellular cyclic nucleotides, e.g., cAMP or cGMP. In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. In one embodiment, the changes in intracellular cAMP or cGMP can be measured using immunoassays. The method described in Offermanns & Simon, J. Bio. Chem., 270:15175-15180 (1995), may be used to determine the level of cAMP. Also, the method described in Felley-Bosco et al., Am. J. Resp. Cell and Mol. Biol., 11:159-164 (1994), may be used to determine the level of cGMP. Further, an assay kit for measuring cAMP and/or cGMP is described in U.S. Pat. No. 4,115,538, herein incorporated by reference.

In another embodiment, transcription levels can be measured to assess the effects of a test compound on signal transduction. A host cell containing T1R polypeptide of interest is contacted with a test compound for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription based assays using a reporter gene may be used as described in U.S. Pat. No. 5,436,128, herein incorporated by reference. The reporter genes can be, e.g., chloramphenicol acetyltransferase, luciferase, beta-galactosidase, beta-lactamase and alkaline phosphatase. Furthermore, the protein of interest can be used as an indirect reporter via attachment to a second reporter such as green fluorescent protein (see, e.g., Mistili & Spector, Nature Biotechnology, 15:961-964 (1997)).

The amount of transcription is then compared to the amount of transcription in either the same cell in the absence of the test compound, or it may be compared with the amount of transcription in a substantially identical cell that lacks the T1R polypeptide(s) of interest. A substantially identical cell may be derived from the same cells from which the recombinant cell was prepared but which had not been modified by introduction of heterologous DNA. Any difference in the amount of transcription indicates that the test compound has in some manner altered the activity of the T1R polypeptide of interest.

### 4. Transgenic Non-Human Animals Expressing Chemosensory Receptors

Non-human animals expressing one or more T1R taste receptor sequences of the invention can also be used for the subject T1R enhancer assays. Such expression can be used to determine whether a test compound specifically binds to a mammalian taste transmembrane receptor complex in vivo by pre-contacting or preincubating a non-human animal stably or transiently transfected with nucleic acids encoding chemosensory receptors or ligand-binding regions thereof with a test potential enhancer compound and thereafter contacting said animal with a known sweet or umami ligand determining whether the animal reacts to the known sweet or umami ligand differently because of the initial preincubation step with the test potential enhancer compound.

Animals transfected or infected with the vectors of the invention are particularly useful for assays to identify and characterize taste stimuli that can bind to a specific or sets of receptors. Such vector-infected animals expressing human taste receptor sequences can be used for in vivo screening of taste stimuli and their effect on, e.g., cell physiology (e.g., on taste neurons), on the CNS, or behavior.

Means to infect/express the nucleic acids and vectors, either individually or as libraries, are well known in the art. A variety of individual cell, organ, or whole animal parameters can be measured by a variety of means. The T1R sequences of the invention can be for example expressed in animal taste tissues by delivery with an infecting agent, e.g., adenovirus expression vector.

The endogenous taste receptor genes can remain functional and wild-type (native) activity can still be present. In other situations, where it is desirable that all taste receptor activity is by the introduced exogenous hybrid receptor, use of a knockout line is preferred. Methods for the construction of non-human transgenic animals, particularly transgenic mice, and the selection and preparation of recombinant constructs for generating transformed cells are well known in the art.

Construction of a “knockout” cell and animal is based on the premise that the level of expression of a particular gene in a mammalian cell can be decreased or completely abrogated by introducing into the genome a new DNA sequence that serves to interrupt some portion of the DNA sequence of the gene to be suppressed. Also, “gene trap insertion” can be used to disrupt a host gene, and mouse embryonic stem (ES) cells can be used to produce knockout transgenic animals (see, e.g., Holzschu, Transgenic Res 6:97-106 (1997)). The insertion of the exogenous is typically by homologous recombination between complementary nucleic acid sequences. The exogenous sequence is some portion of the target gene to be modified, such as exonic, intronic or transcriptional regulatory sequences, or any genomic sequence which is able to affect the level of the target gene's expression; or a combination thereof. Gene targeting via homologous recombination in pluripotential embryonic stem cells allows one to modify precisely the genomic sequence of interest. Any technique can be used to create, screen for, propagate, a knockout animal, e.g., see Bijvoet, Hum. Mol. Genet. 7:53-62 (1998); Moreadith, J. Mol. Med. 75:208-216 (1997); Tojo, Cytotechnology 19:161-165 (1995); Mudgett, Methods Mol. Biol. 48:167-184 (1995); Longo, Transgenic Res. 6:321-328 (1997); U.S. Pat. Nos. 5,616,491; 5,464,764; 5,631,153; 5,487,992; 5,627,059; 5,272,071; WO 91/09955; WO 93/09222; WO 96/29411; WO 95/31560; WO 91/12650.

The nucleic acids of the invention can also be used as reagents to produce “knockout” human cells and their progeny. Likewise, the nucleic acids of the invention can also be used as reagents to produce “knock-ins” in mice. The human or rat T1R gene sequences can replace the orthologs T1R in the mouse genome. In this way, a mouse expressing a human or rat T1R is produced. This mouse can then be used to analyze the function of human or rat T1Rs, and to identify ligands for such T1Rs.

### Modulators

The compounds tested as modulators (preferably enhancers) of a T1R family member can be any small chemical compound, or a biological entity, such as a protein, sugar, nucleic acid or lipid. Alternatively, modulators can be genetically altered versions of a T1R family member. Typically, test compounds may be small chemical molecules and peptides. Essentially any chemical compound can be used as a potential modulator or ligand in the assays of the invention, although most often compounds can be dissolved in aqueous or organic (especially DMSO-based) solutions are used. The assays may be designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual consumer products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res., 37:487-93 (1991) and Houghton et al., Nature, 354:84-88 (1991)). Other chemistries for generating chemical diversity libraries can also be used. Such chemistries include, but are not limited to: peptoids (e.g., WO 91/19735), encoded peptides (e.g., WO 93/20242), random bio-oligomers (e.g., WO 92/00091), benzodiazepines (e.g., U.S. Pat. No. 5,288,514), diversomers such as hydantoins, benzodiazepines and dipeptides (Hobbs et al., PNAS., 90:6909-13 (1993)), vinylogous polypeptides (Hagihara et al., J. Amer. Chem. Soc., 114:6568 (1992)), nonpeptidal peptidomimetics with glucose scaffolding (Hirschmann et al., J. Amer. Chem. Soc., 114:9217-18 (1992)), analogous organic syntheses of small compound libraries (Chen et al., J. Amer. Chem. Soc., 116:2661 (1994)), oligocarbamates (Cho et al., Science, 261:1303 (1993)), peptidyl phosphonates (Campbell et al., J. Org. Chem., 59:658 (1994)), nucleic acid libraries (Ausubel, Berger, and Sambrook, all supra), peptide nucleic acid libraries (U.S. Pat. No. 5,539,083), antibody libraries (Vaughn et al., Nature Biotechnology, 14(3):309-14 (1996) and PCT/US96/10287), carbohydrate libraries (Liang et al., Science, 274:1520-22 (1996) and U.S. Pat. No. 5,593,853), small organic molecule libraries (benzodiazepines, Baum, C&EN, January 18, page 33 (1993); thiazolidinones and metathiazanones, U.S. Pat. No. 5,549,974; pyrollidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS (Advanced Chem Tech, Louisville Ky.), Symphony (Rainin, Woburn, Mass.), 433A (Applied Biosystems, Foster City, Calif.), 9050 Plus (Millipore, Bedford, Mass.)). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences; Columbia, Md.; etc.).

In one aspect of the invention, the T1R modulators can be used in any food product, confectionery, pharmaceutical composition, or ingredient thereof to thereby modulate the taste of the product, composition, or ingredient in a desired manner. For instance, T1R modulators that elicit sweet or umami taste sensation can be added to provide an improved sweet or umami taste to a product or composition, while T1R modulators which enhance sweet or umami taste sensations can be added to enhance the sweet or umami taste of another compound in a composition such as a food or beverage product or composition. Also, the invention provides means of identifying sweet or umami compounds and enhancers found in foods, beverages and medicinals and producing taste improved foods, beverages and medicinals lacking or having a reduced quantity thereof. In addition they are useful as therapeutics as afore-described e.g., in treating and managing obesity, diabetes, weight, and conditions relating to glucose, insulin, and fat metabolism, transport, release, and absorption.

### Use of Compounds Identified by the Invention

T1R enhancer compounds identified according to the invention may be added to foods, beverages or medicinal compositions to modulate sweet or umami taste.

As noted previously, preferably, the taste modulatory properties of compounds identified in the subject cell-based assays may be confirmed in taste tests, e.g., human or non-human animal taste tests.

### Kits

The subject assays or inhibitors which use T1R genes and their homologs are useful tools for identifying T1R enhancers as well as tools for identifying taste receptor cells, for forensics and paternity determinations, and for examining taste transduction. T1R family member-specific reagents that specifically hybridize to T1R nucleic acids, such as T1R probes and primers, and T1R specific reagents that specifically bind to a T1R protein, e.g., T1R antibodies are used to examine taste cell expression and taste transduction regulation.

Nucleic acid assays for the presence of DNA and RNA for a T1R family member in a sample include numerous techniques are known to those skilled in the art, such as southern analysis, northern analysis, dot blots, RNase protection, SI analysis, amplification techniques such as PCR, and in situ hybridization. In in situ hybridization, for example, the target nucleic acid is liberated from its cellular surroundings in such as to be available for hybridization within the cell while preserving the cellular morphology for subsequent interpretation and analysis. The following articles provide an overview of the art of in situ hybridization: Singer et al., Biotechniques, 4:230250 (1986); Haase et al., Methods in Virology, vol. VII, 189-226 (1984); and Names et al., eds., Nucleic Acid Hybridization: A Practical Approach (1987). In addition, a T1R protein can be detected with the various immunoassay techniques described above. The test sample is typically compared to both a positive control (e.g., a sample expressing a recombinant T1R protein) and a negative control.

The present invention therefore also provides for kits for screening for modulators (enhancers or inhibitors) of T1R family members. Such kits can be prepared from readily available materials and reagents. For example, such kits can comprise any one or more of the following materials: T1R nucleic acids or proteins, reaction tubes, and instructions for testing T1R activity. Optionally, the kit contains a functional T1R polypeptide. A wide variety of kits and components can be prepared according to the present invention, depending upon the intended user of the kit and the particular needs of the user.

Having now generally described the invention, the same will be more readily understood by reference to the following examples, which are provided by way of illustration and are not intended as limiting. It is understood that various modifications and changes can be made to the herein disclosed exemplary embodiments without departing from the spirit and scope of the invention.

### EXAMPLES

### Materials and Methods Used in Examples

As described supra, novel cell based assay conditions, using the human sweet receptor (T1R2 and T1R3) expressed in HEK293 cells, were developed and used for the identification of hT1R2/hT1R3 enhancers or inhibitors (modulators). Particularly hT1R2/hT1R3-HEK293 Galpha₁₅ cells were seeded in 384-well-clear bottom plates (Fisher) at a density of ˜50,000 cells/well and grown overnight at 37 C. On the day of the experiment, cells were loaded with the calcium indicator Fluo3AM (2 uM) (Invitrogen) in D-PBS (Invitrogen) using a Multidrop (Titertek). Cells were incubated for 1 hour at room temperature and excess dye was washed out with D-PBS using an EMBLA cell washer (Molecular Devices), leaving a residual volume of 25 □l/well. After 30 minutes of rest time at room temperature, Fluo3AM-loaded cell plate, a compound plate and a sweetener plate were loaded into a Fluorometric Imaging Plate Reader (FLIPR, Molecular Devices). 384-well compound plate and the sweetener plate were prepared at 3× final concentration in D-PBS. Imaging was initiated with the acquisition of the baseline fluorescence for a period of 7 to 10 seconds, and then cells were stimulated on line with addition of 25 □l/well of the compound plate (pre-incubation step). Cells were incubated for 7.5 minutes with the compound prior a last stimulation with the sweetener plate.

Using this protocol a novel modulator, S2423, was discovered which enhances the Sucralose potency on the sweet receptor by about 4 to 5 fold at 25 uM. By contrast, S2423 does not enhance the effect of other sweeteners such as D-Fructose, Sucrose, Aspartame and also does not enhance the effects of two other un-related GPCR agonists Isoproterenol and carbachol. Other experiments described in the Examples below suggest that the novel pre-incubation step is important to detect the enhancement effect of S2423 (and potentially other T1R enhancer compounds) as co-stimulation of the cells with a known sweetener and the S2423 enhancer compound does not reveal similar magnitude of enhancement.

### Example 1

### Novel Assay Conditions and Identification of a Sweet Receptor Enhancer.

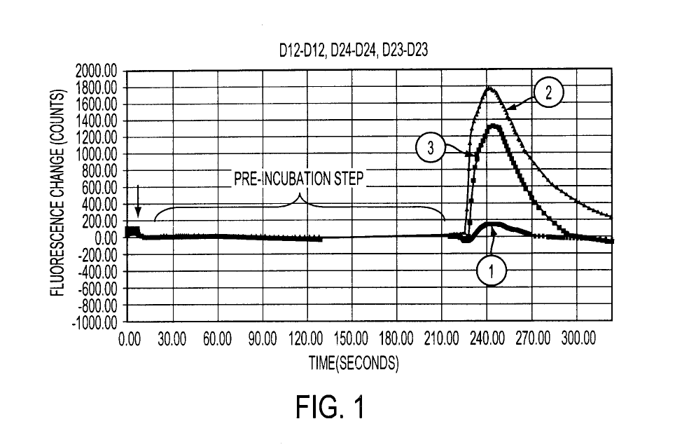

In the experiment contained in FIG. 1, HEK293 cells expressing the human Sweet receptor and G□15 were loaded with Fluo3AM. Cells were stimulated with 50 uM S2423 (arrow) and incubated for 7.5 minutes (pre-incubation step). A suboptimal concentration of Sucralose (Trace #1) was then added to the wells of the plate. It can be seen in the Figure that the well that had been pre-incubated with S2423 showed a much greater response (Trace #3) that corresponded to about ˜70% of the maximum receptor response obtained with a saturating concentration of Sucralose (Trace #2).

### Example 2

### Effect of Increasing Concentrations of S2423.

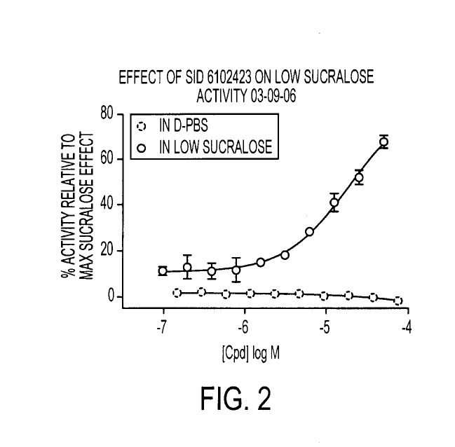

In the experiment contained in FIG. 2, HEK293 cells expressing the human Sweet receptor and Galpha15 were loaded with Fluo3AM and tested using a stimulation protocol similar to the one described in FIG. 1. In this case, however, cells were pre-incubated with increasing concentrations of S2423 and then stimulated with D-PBS or a low concentration of Sucralose. At each concentration tested, it can be seen in the Figure that the identified enhancer compound S2423 becomes only active in the presence of Sucralose, one of the hallmarks for a receptor enhancer.

### Example 3

### Effect of S2423 on Sucralose Dose-Response in the Assay.

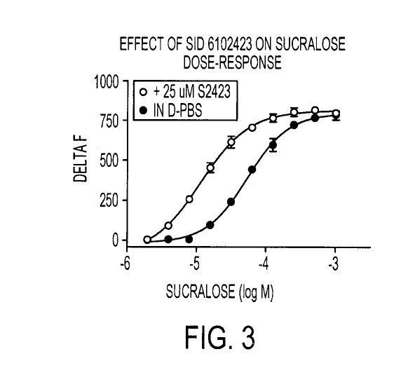

In the experiment which is contained in FIG. 3, HEK293 cells expressing the human sweet receptor and Galpha15 were again loaded with Fluo3AM and tested using a stimulation protocol similar to the one described in FIG. 1. In this case, however, cells were pre-incubated with a fixed concentration of S2423 (50 uM or 25 uM final) and then stimulated with increasing concentrations of Sucralose. As shown in FIG. 3, the identified enhancer compound, S2423, increases the potency of Sucralose in the assay by ˜4-5 fold (from 53 uM to 11 uM).

### Example 4

### Effect of S2423 on Sweeteners and GPCR Agonists Dose-Response in the Assay.

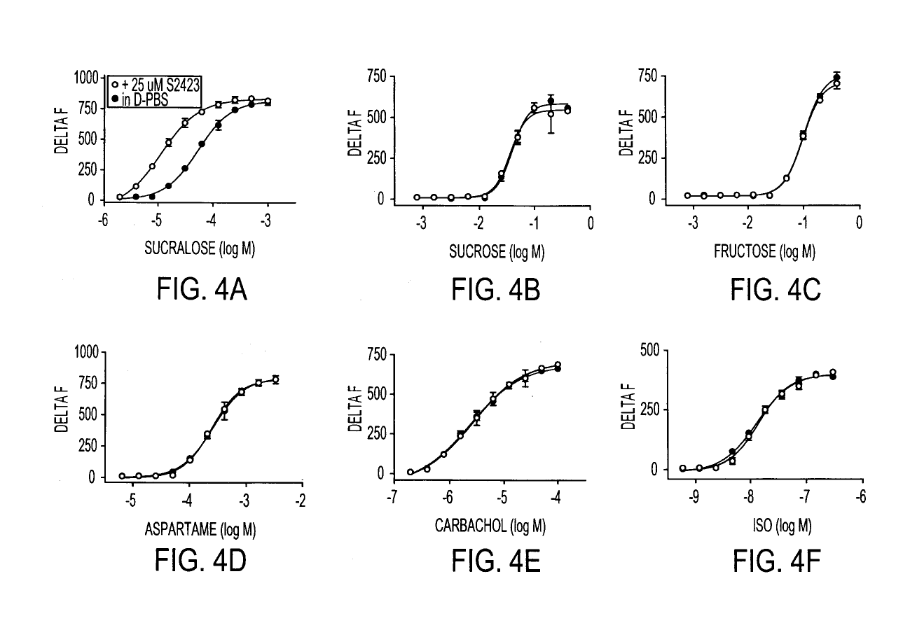

In this experiment the results of which are contained in FIG. 4, HEK293 cells expressing the human Sweet receptor and Galpha15 were again loaded with Fluo3AM and tested using a stimulation protocol similar to the one described in FIG. 1. In this case, however, cells were pre-incubated with a fixed concentration of S2423 (50 uM or 25 uM final) and then stimulated with increasing concentrations of Sucralose, D-Fructose, Sucrose, Aspartame, Carbachol and Isoproterenol. As can be seen from the results contained in the Figure, the S2423 enhancer compound specifically increases the potency of Sucralose in the assay. By contrast it shows no effect on the other sweeteners and un-related GPCR agonists. Therefore it is a specific enhancer of Sucralose.

### Example 5

### S2423 Requires Pre-Incubation for Enhancement.

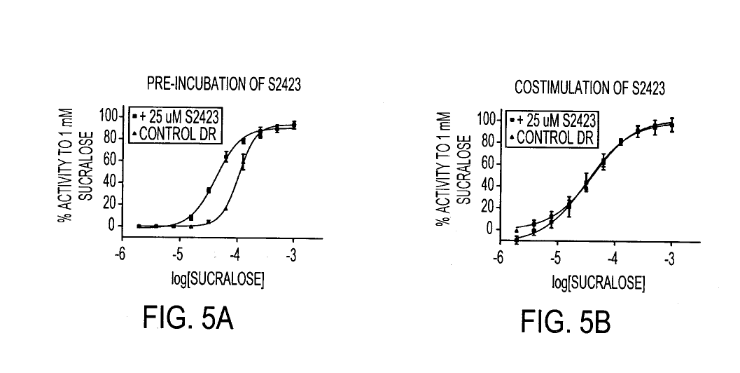

In the experiment the results of which are contained in FIG. 5, HEK293 cells expressing the human Sweet receptor and Galpha15 were again loaded with Fluo3AM. The left panel describes results obtained when performing an experiment identical to the one described in FIG. 3. The right panel describes results obtained when S2423 was added simultaneously with the sweetener (instead of using a pre-incubation protocol). It can be seen therefrom that S2423 enhancement can only be picked up if there is a pre-incubation step as described.

All of the references cited herein are incorporated by reference in their entirety. Having described the invention, the following claims are provided below.

We claim:

1. An assay method for identifying a modulator (enhancer or inhibitor) of an activator the sweet (T1R2/T1R3) or umami (T1R1/T1R3) taste receptor comprising
(i) preincubating a sweet receptor or umami receptor respectively comprised of T1R2 and T1R3 polypeptides or T1R1 and T1R3 polypeptides with at least one first compound that is being screened as a potential modulator of said sweet or umami receptor;
(ii) after said preincubation step contacting said preincubated sweet or umami receptor with a suboptimal concentration of at least one second compound that activates said sweet or umami receptor, wherein a suboptimal concentration of said second compound is a concentration below the saturation concentration that results in maximal detectable activity of the sweet or umami receptor;
(iii) detecting sweet or umami receptor activity after step (ii) and comparing said activity to sweet or umami receptor activity that is detected when said sweet or umami receptor is contacted with said suboptimal concentration of said at least one second compound that activates the sweet or umami receptor in the absence of said preincubation step with said at least one first compound;
(iv) identifying the screened first compound as a putative enhancer or inhibitor of said second compound on the activation of the sweet or umami receptor if the detected sweet or umami receptor activity is respectively greater or lesser in the presence of the preincubation step (ii) than in the absence of said preincubation step (ii).

2. The assay of claim 1 wherein the sweet or umami receptor comprises hT1R2/hT1R3, rT1R2/rT1R3, or mT1R2/mT1R3.

3. The assay of claim 1 wherein the sweet or umami receptor comprises a chimeric T1R1, T1R2 or T1R3 polypeptide.

4. The assay of claim 1 wherein a positive enhancer or inhibitor compound is further evaluated in human or animal taste tests to confirm its effect on taste.

5. The assay of claim 1 wherein said second compound is a sweet or umami ligand.

6. The assay of claim 5 wherein said sweet or umami ligand is a naturally occurring sweet or umami compound.

7. The assay of claim 6 wherein said compound is selected from sucrose, glucose, fructose, lactose, and mannose.

8. The assay of claim 5 wherein said sweet or umami ligand is synthetic.

9. The assay of claim 9 wherein said synthetic ligand is selected from Sucralose, saccharin, aspartame, monellin, acesulfame K, cyclamate, 8-chlorophenylthio-adenosine 3′,5′ cyclic monophosphate (8-cpt-cAMP) and dibutyryl-guanosine 3′,5′-cyclic monophosphate (db-cGMP).

10. The assay of claim 1 wherein sweet or umami receptor activity is detected by a calcium imaging assay.

11. The assay of claim 10 wherein the assay uses a dye that detects changes in intracellular calcium fluorimetrically.

12. The assay of claim 10 wherein the assay includes use of a calcium indicator selected from Fluo-, Fluo-4, fura-2, indo-1, quin-2, oregon green, calcium green 2, or a calcium sensitive protein.

13. The assay of claim 12 wherein the calcium sensitive protein is aequorin, apo-aequorin, or luciferase.

14. The assay of claim 12 wherein the calcium indicator is Fluo3AM

15. The assay of claim 11 wherein said calcium specific indicator is loaded onto said T1R2/T1R3 or T1R1/TR3 expressing cells prior to the cells being preincubated with said potential enhancer compound.

16. The assay of claim 10 which detects changes in intracellular calcium in eukaryotic T1R2/T1R3 or T1R1/T1R3 expressing cells.

17. The assay of claim 16 wherein said eukaryotic cells are mammalian, insect, yeast, avian or amphibian cells.

18. The assay of claim 15 wherein the eukaryotic cells are selected from HEK-293, COS, CHO, and BHK cells.

19. The assay of claim 18 wherein the cells are HEK-293 cells.

20. The assay of claim 16 wherein the T1R2/T1R3 or T1R1/T1R3 expressing eukaryotic cell expresses a G protein that functionally couples to said T1R2/T1R3 or T1R1/T1R3 receptor polypeptides.

21. The assay of claim 20 wherein the G protein is selected from Galpha15, Galpha16, gustducin, transducin, and chimeras thereof.

22. The assay of claim 1 wherein the suboptimal concentration of the sweet or umami ligand results is at most 10-50% of the saturation concentration of the sweet or umami ligand which results in maximal detectable activation of the sweet or umami receptor.

23. The assay of claim 22 wherein the suboptimal concentration of said sweet or umami ligand is at most 10-25% of the saturation concentration of the sweet or umami ligand that results in maximal detectable activation of the sweet or umami receptor.

24. The assay of claim 10 which comprises use of a fluorimetric imaging plate reader (FLIPR) or a voltage imaging plate reader (VIPR).

25. The assay of claim 10 wherein the sweet ligand is Sucralose.

26. The assay of claim 1 wherein the T1R2/T1R3 sweet or T1R1/T1R3 umami taste receptor is contained on a cell membrane.

27. The assay of claim 1 wherein the T1R2/T1R3 sweet or T1R1/T1R3 umami taste receptor is expressed by a cell.

28. The assay of claim 27 wherein said cell is a mammalian cell or an oocyte.

29. The assay of claim 28 which is a fluorimetric assay.

30. The assay of claim 28 which is an electrophysiological assay.

31. The assay of claim 1 which is a binding assay.

32. The assay of claim 1 wherein the T1R2/T1R3 sweet or T1R1/T1R3 umami taste receptor is directly or indirectly covalently or non-covalently attached to a solid phase.

33. The assay of claim 32 which is a binding assay.

34. The assay of claim 1 which detects the effect of said putative enhancer or inhibitor compound on an intracellular ion.

35. The assay of claim 34 wherein said ion is calcium.

36. The assay of claim 35 wherein the effect of said compound on calcium is detected using a membrane sensitive dye or a voltage sensitive dye.

37. The assay of claim 36 wherein the readout is fluorimetric

38. The assay of claim 1 which detects the effect of said putative enhancer or inhibitor compound on ion polarization.

39. The assay of claim 1 which detects the effect of said potential enhancer or inhibitor compound on second messenger levels.

40. The assay of claim 39 wherein the second messenger is IP3.

41. The assay of claim 40 which detects the effect of said putative enhancer or inhibitor compound on intracellular cyclic nucleotides.

42. The assay of claim 41 wherein said nucleotides are cGMP or cAMP.

43. The assay of claim 1 which detects the effect of said potential enhancer or inhibitor compound on G protein binding to GTPγS.

44. The assay of claim 10 wherein the T1R2/T1R3 expressing cells are seeded into wells comprised in a multiwell containing plate.

45. The assay of claim 44 wherein the plate contains about 24, 36, 48, 64, 72, 96, 128, 192, 256 or 384 wells.

46. The assay of claim 1 wherein the preincubation step is effected for about 1-10 minutes prior to contacting the T1R2/T1R3 or T1R1/T1R3 receptor with the suboptimal concentration of the sweet or umami ligand.

47. The assay of claim 1 wherein the identified enhancer or inhibitor is evaluated for potential as a therapeutic for treating a condition selected from diabetes, obesity, weight control, fat metabolism, glucose metabolism, glucose release and/or transport, insulin release and/or insulin metabolism, satiety and/or the release of satiety peptide.

48. The assay of claim 47 wherein the satiety peptide is GLP-1.

49. The assay of claim 47 wherein the glucose transporter is GLUT2 or SGLT1.

50. The assay of any one of claim 47 wherein the assays uses endogenous cells which express T1R2/T1R3 or T1R1/T1R3.

51. The assay of claim 50 wherein said cells are taste cells comprised on the tongue, oral cavity, or in the gastrointestinal tract and associated organs including pancreas, liver, intestines, stomach, and gall bladder.

52. The assay of claim 51 wherein the cells comprise gastroendocrine cells or taste bud cells.

53. The assay of claim 47 wherein the identified compound does not elicit an effect on sweet or umami taste.
